(19) United States
(12) Patent Application Publication
(10) Pub. No.: US 2018/0104236 A1
(43) Pub. Date: Apr. 19, 2018

(54) METHODS OF TREATMENT COMPRISING ADMINISTERING A HIGH DAILY DOSE OF OXYCODONE AND NALOXONE IN A 2:1 WEIGHT RATIO

(71) Applicant: Euro-Celtique S. A., Luxembourg (LU)

(72) Inventors: Michael HOPP, Bad Camberg (DE); Petra Leyendecker, Braunfels (DE); Wolfram Kremers, Bad Honnef (DE)

(21) Appl. No.: 15/716,098

(22) Filed: Sep. 26, 2017

Related U.S. Application Data
(60) Provisional application No. 62/462,772, filed on Feb. 23, 2017.
(60) Provisional application No. 62/399,968, filed on Sep. 26, 2016.

(51) Int. Cl. A61K 31/485; A61P 1/10; A61P 25/04

(52) U.S. Cl. CPC A61K 31/485; A61K 2300/00; A61P 25/04; A61P 1/10

(57) ABSTRACT

The present invention relates to methods of treatment comprising, inter alia, administering a high daily dose of oxycodone, such as a daily dose of at least 90 mg oxycodone, and naloxone in a 2:1 weight ratio to a patient in need thereof.

### TECHNICAL FIELD OF THE INVENTION

The present invention relates to methods of treatment comprising, inter alia, administering a high daily dose of oxycodone, such as a daily dose of at least 90 mg oxycodone, and naloxone in a 2:1 weight ratio to a patient in need thereof.

### BACKGROUND OF THE INVENTION

Oxycodone/naloxone prolonged release (OXN PR) is an oral prolonged-release formulation containing oxycodone and naloxone in a 2:1 ratio. This fixed combination has been shown to provide sufficient analgesic effect of oxycodone but with an improved safety profile (Meissner et al., Eur. J. Pain, 13, 56-64 (2009)). The product was released in Germany in 2006 and is now available, inter alia, in different European countries in different dose strengths for twice-daily use. OXN PR uses the opioid antagonist naloxone to maintain bowel function by counteracting opioid-induced constipation (OIC) by blocking the action of oxycodone at opioid-receptors locally in the gut. For many years, the highest dosage strength approved by regulatory authorities was the twice-daily use of the OXN 40/20 PR tablet resulting in daily doses of oxycodone of 80 mg and of naloxone of 40 mg (OXN 80/40 PR).

However, it was found that when a patient's pain was very strong, there was a need for daily doses higher than the above-mentioned maximum approved daily dose of OXN 80/40 PR. In order to achieve sufficient analgesia for such situations, patients were often instructed to take a daily dose of up to 400 mg oxycodone prolonged release (OxyPR) in addition to the maximum OXN dose.

Physicians considered that this extra dosing of oxycodone might impair the beneficial bowel function and OIC effects of OXN, and so there remained a need for providing higher levels of analgesia that did not lead to a worsening of bowel function or OIC. Unfortunately, initial results of the study by Meissner et al., 2009 were interpreted as pointing towards a decrease of OXN's positive bowel function and OIC effects if daily doses higher than OXN 80/40 would be administered (see in particular FIG. 6 of Meissner et al., 2009). This meant that patients suffering from intense pain and in need of large daily doses of opioid analgesics would be unable to use such treatment regimens without suffering from loss of bowel function and debilitating OIC.

Cancer patients frequently suffer from various bowel dysfunction symptoms—such as abdominal discomfort, nausea, vomiting, and especially constipation—that are a result of their disease state and associated cancer treatment. The intense pain from cancer often leaves such patients in need of the strong pain relief afforded by opioid analgesics, but studies have demonstrated that such patients often forego adequate pain relief and endure a lower quality of life because of the disruption in bowel function and OIC associated with opioids (Cuomo et al., Am. J. Hosp. Palliat. Med., 31(8) 867-876 (2014) (1998)). Cancer patients are more susceptible to opioid-induced bowel dysfunction and constipation at least because they are already experiencing bowel dysfunction from their cancer and cancer treatment and because they typically require high daily doses of strong opioids, and such high daily doses were known to produce more bowel dysfunction and OIC than lower daily doses (Sykes, N. P., Palliat. Med., 12, 375-382 (1998)).

The present disclosure has surprisingly found that the potential worsening of bowel function and OIC at daily OXN doses higher than 80/40 does not occur and that such higher OXN amounts can be administered to patients in need of high levels of pain relief, such as cancer patients, while still providing a clinically significant effect on their bowel function.

### OBJECTS AND SUMMARY OF THE INVENTION

It is an object of certain embodiments of the present invention to provide methods of improving bowel function, such as reducing or preventing opioid-induced constipation.

The above object and others can be achieved by the present invention, which in certain embodiments is directed to a method of improving bowel function comprising orally administering to a patient in need thereof a daily dose comprising oxycodone or a pharmaceutically acceptable salt thereof and naloxone or a pharmaceutically acceptable salt thereof in a 2:1 weight ratio;

wherein the daily dose comprises at least 90 mg of the oxycodone or pharmaceutically acceptable salt thereof; and

wherein the daily dose is administered as two or more prolonged release oral dosage forms.

It is an object of certain embodiments of the present invention to provide methods of treating pain and improving bowel function, such as reducing or preventing opioid-induced constipation.

The above object and others can be achieved by the present invention, which in certain embodiments is directed to a method of treating pain and improving bowel function comprising orally administering to a patient in need thereof a daily dose comprising oxycodone or a pharmaceutically acceptable salt thereof and naloxone or a pharmaceutically acceptable salt thereof in a 2:1 weight ratio;

wherein the daily dose comprises at least 90 mg of the oxycodone or pharmaceutically acceptable salt thereof; and

wherein the daily dose is administered as two or more prolonged release oral dosage forms.

It is an object of certain embodiments of the present invention to provide methods of maintaining bowel function in a patient on opioid therapy.

The above object and others can be achieved by the present invention, which in certain embodiments is directed to a method of maintaining bowel function in a patient on opioid therapy comprising orally administering to the patient a daily dose comprising oxycodone or a pharmaceutically acceptable salt thereof and naloxone or a pharmaceutically acceptable salt thereof in a 2:1 weight ratio;

wherein the daily dose comprises at least 90 mg of the oxycodone or pharmaceutically acceptable salt thereof; and

wherein the daily dose is administered as two or more prolonged release oral dosage forms.

It is an object of certain embodiments of the present invention to provide methods of treating pain and maintaining bowel function.

The above object and others can be achieved by the present invention, which in certain embodiments is directed to a method of treating pain and maintaining bowel function in a patient comprising orally administering to the patient a daily dose comprising oxycodone or a pharmaceutically acceptable salt thereof and naloxone or a pharmaceutically acceptable salt thereof in a 2:1 weight ratio;

wherein the daily dose comprises at least 90 mg of the oxycodone or pharmaceutically acceptable salt thereof; and

wherein the daily dose is administered as two or more prolonged release oral dosage forms.

It is an object of certain embodiments of the present invention to provide methods of treating breakthrough pain in a patient on opioid therapy.

The above object and others can be achieved by the present invention, which in certain embodiments is directed to a method of treating breakthrough pain in a patient on opioid therapy while maintaining bowel function comprising orally administering to the patient immediate release oxycodone without an opioid antagonist;

wherein the opioid therapy comprises orally administering to the patient a daily dose comprising oxycodone or a pharmaceutically acceptable salt thereof and naloxone or a pharmaceutically acceptable salt thereof in a 2:1 weight ratio;

wherein the daily dose comprises at least 90 mg of the oxycodone or pharmaceutically acceptable salt thereof;

wherein the daily dose is administered as two or more prolonged release oral dosage forms.

It is an object of certain embodiments of the present invention to provide methods for treating breakthrough pain in a patient while treating chronic pain.

The above object and others can be achieved by the present invention, which in certain embodiments is directed to a method of treating breakthrough pain in a patient while treating chronic pain and maintaining bowel function in the patient comprising orally administering to the patient immediate release oxycodone without an opioid antagonist for treating breakthrough pain;

wherein the chronic pain is treated by orally administering to the patient a daily dose comprising oxycodone or a pharmaceutically acceptable salt thereof and naloxone or a pharmaceutically acceptable salt thereof in a 2:1 weight ratio;

wherein the daily dose comprises at least 90 mg of the oxycodone or pharmaceutically acceptable salt thereof;

wherein the daily dose is administered as two or more prolonged release oral dosage forms.

It is an object of certain embodiments of the present invention to provide methods for normalizing bowel function in a patient on opioid therapy.

The above object and others can be achieved by the present invention, which in certain embodiments is directed to a method of normalizing bowel function in a patient on opioid therapy comprising orally administering to the patient a daily dose comprising oxycodone or a pharmaceutically acceptable salt thereof and naloxone or a pharmaceutically acceptable salt thereof in a 2:1 weight ratio;

wherein the daily dose comprises at least 90 mg of the oxycodone or pharmaceutically acceptable salt thereof; and

wherein the daily dose is administered as two or more prolonged release oral dosage forms.

It is an object of certain embodiments of the present invention to provide methods for treating pain and normalizing bowel function in a patient.

The above object and others can be achieved by the present invention, which in certain embodiments is directed to a method of treating pain and normalizing bowel function in a patient comprising orally administering to the patient a daily dose comprising oxycodone or a pharmaceutically acceptable salt thereof and naloxone or a pharmaceutically acceptable salt thereof in a 2:1 weight ratio;

wherein the daily dose comprises at least 90 mg of the oxycodone or pharmaceutically acceptable salt thereof; and

wherein the daily dose is administered as two or more prolonged release oral dosage forms.

It is an object of certain embodiments of the present invention to provide methods for treating pain in a patient suffering from cancer.

The above object and others can be achieved by the present invention, which in certain embodiments is directed to a method of treating pain in a patient suffering from cancer comprising orally administering to the patient a daily dose comprising oxycodone or a pharmaceutically acceptable salt thereof and naloxone or a pharmaceutically acceptable salt thereof in a 2:1 weight ratio;

wherein the daily dose comprises at least 90 mg of the oxycodone or pharmaceutically acceptable salt thereof; and

wherein the daily dose is administered as two or more prolonged release oral dosage forms.

The above embodiments disclose their subject matter as “methods of treatment”. It is noted that the corresponding subject matter may also be formulated differently, e.g. as “for use in treating”. The “for use in treating” wording is given for the subject matter of the above embodiments in the following (it is noted that such a wording may of course also be used for the subject matter of all further embodiments given in this application below):

A daily dose comprising at least 90 mg of oxycodone or a pharmaceutically acceptable salt thereof and naloxone or a pharmaceutical acceptable salt thereof in a 2:1 weight ratio for use in improving bowel function, wherein the daily dose is administered orally and wherein the daily dose is administered as two or more prolonged release oral dosage forms.

A daily dose comprising at least 90 mg of oxycodone or a pharmaceutically acceptable salt thereof and naloxone or a pharmaceutical acceptable salt thereof in a 2:1 weight ratio for use in treating pain and improving bowel function, wherein the daily dose is administered orally and wherein the daily dose is administered as two or more prolonged release oral dosage forms.

A daily dose comprising at least 90 mg of oxycodone or a pharmaceutically acceptable salt thereof and naloxone or a pharmaceutical acceptable salt thereof in a 2:1 weight ratio for use in maintaining bowel function in a patient on opioid therapy, wherein the daily dose is administered orally and wherein the daily dose is administered as two or more prolonged release oral dosage forms.

A daily dose comprising at least 90 mg of oxycodone or a pharmaceutically acceptable salt thereof and naloxone or a pharmaceutical acceptable salt thereof in a 2:1 weight ratio for use in treating pain and maintaining bowel function, wherein the daily dose is administered orally and wherein the daily dose is administered as two or more prolonged release oral dosage forms.

Immediate release oxycodone for use in treating breakthrough pain in a patient on opioid therapy while maintaining bowel function, wherein the immediate release oxycodone is administered without an opioid antagonist, wherein the opioid therapy comprises orally administering a daily dose comprising at least 90 mg of oxycodone or a pharmaceutically acceptable salt thereof and naloxone or a pharmaceutical acceptable salt thereof in a 2:1 weight ratio, and wherein the daily dose is administered as two or more prolonged release oral dosage forms.

Immediate release oxycodone for use in treating breakthrough pain while treating chronic pain and maintaining bowel function, wherein the immediate release oxycodone is administered without an opioid antagonist, wherein the chronic pain is treated by orally administering a daily dose comprising at least 90 mg of oxycodone or a pharmaceutically acceptable salt thereof and naloxone or a pharmaceutical acceptable salt thereof in a 2:1 weight ratio, and wherein the daily dose is administered as two or more prolonged release oral dosage forms.

A daily dose comprising at least 90 mg of oxycodone or a pharmaceutically acceptable salt thereof and naloxone or a pharmaceutical acceptable salt thereof in a 2:1 weight ratio for use in normalizing bowel function in a patient on opioid therapy, wherein the daily dose is administered orally and wherein the daily dose is administered as two or more prolonged release oral dosage forms.

A daily dose comprising at least 90 mg of oxycodone or a pharmaceutically acceptable salt thereof and naloxone or a pharmaceutical acceptable salt thereof in a 2:1 weight ratio for use in treating pain and normalizing bowel function, wherein the daily dose is administered orally and wherein the daily dose is administered as two or more prolonged release oral dosage forms.

A daily dose comprising at least 90 mg of oxycodone or a pharmaceutically acceptable salt thereof and naloxone or a pharmaceutical acceptable salt thereof in a 2:1 weight ratio for use in treating pain in a patient suffering from cancer, wherein the daily dose is administered orally and wherein the daily dose is administered as two or more prolonged release oral dosage forms.

The methods disclosed herein may also be understood as the corresponding “use in the manufacture of a medicament” (so-called “Swiss-type” language).

### BRIEF DESCRIPTION OF THE DRAWINGS

FIG. 1 is a graphical depiction of the study design of Example 1.

FIG. 2 shows the consort flow diagram.

FIG. 3A and FIG. 3B show the results of the BFI in the full analysis populations for (A) the total study group and (B) the cancer subgroup.

FIG. 4A and FIG. 4B show the pain scores for (A) the total study group and (B) the cancer subgroup.

FIG. 5 shows the Bowel Function Index in the extension phase described in Example 2—Observed Values: Total Exposure Safety Population.

FIG. 6 shows the Bowel Function Index in the extension phase described in Example 2—Observed Values by Core Phase Treatment: Total Exposure Safety Population.

FIG. 7 shows the average pain over the last 24 hours in the extension phase described in Example 2—Observed Values: Total Exposure Safety Population.

### DETAILED DESCRIPTION

In certain embodiments, the present disclosure provides a method of improving bowel function comprising orally administering to a patient in need thereof a daily dose comprising oxycodone or a pharmaceutically acceptable salt thereof and naloxone or a pharmaceutically acceptable salt thereof in a 2:1 weight ratio;

wherein the daily dose comprises at least 90 mg of the oxycodone or pharmaceutically acceptable salt thereof; and

wherein the daily dose is administered as two or more prolonged release oral dosage forms.

In some embodiments, the present disclosure provides a method of treating pain and improving bowel function comprising orally administering to a patient in need thereof a daily dose comprising oxycodone or a pharmaceutically acceptable salt thereof and naloxone or a pharmaceutically acceptable salt thereof in a 2:1 weight ratio;

wherein the daily dose comprises at least 90 mg of the oxycodone or pharmaceutically acceptable salt thereof; and

wherein the daily dose is administered as two or more prolonged release oral dosage forms.

In certain embodiments, the present disclosure provides a method of maintaining bowel function in a patient on opioid therapy comprising orally administering to the patient a daily dose comprising oxycodone or a pharmaceutically acceptable salt thereof and naloxone or a pharmaceutically acceptable salt thereof in a 2:1 weight ratio;

wherein the daily dose comprises at least 90 mg of the oxycodone or pharmaceutically acceptable salt thereof; and

wherein the daily dose is administered as two or more prolonged release oral dosage forms.

In some embodiments, the present disclosure provides a method of treating pain and maintaining bowel function in a patient comprising orally administering to the patient a daily dose comprising oxycodone or a pharmaceutically acceptable salt thereof and naloxone or a pharmaceutically acceptable salt thereof in a 2:1 weight ratio;

wherein the daily dose comprises at least 90 mg of the oxycodone or pharmaceutically acceptable salt thereof; and

wherein the daily dose is administered as two or more prolonged release oral dosage forms.

In certain embodiments, the present disclosure provides a method of treating breakthrough pain in a patient on opioid therapy while maintaining bowel function comprising orally administering to the patient immediate release oxycodone without an opioid antagonist;

wherein the opioid therapy comprises orally administering to the patient a daily dose comprising oxycodone or a pharmaceutically acceptable salt thereof and naloxone or a pharmaceutically acceptable salt thereof in a 2:1 weight ratio;

wherein the daily dose comprises at least 90 mg of the oxycodone or pharmaceutically acceptable salt thereof;

wherein the daily dose is administered as two or more prolonged release oral dosage forms.

In some embodiments, the present disclosure provides a method of treating breakthrough pain in a patient while treating chronic pain and maintaining bowel function in the patient comprising orally administering to the patient immediate release oxycodone without an opioid antagonist for treating breakthrough pain;

wherein the chronic pain is treated by orally administering to the patient a daily dose comprising oxycodone or a pharmaceutically acceptable salt thereof and naloxone or a pharmaceutically acceptable salt thereof in a 2:1 weight ratio;

wherein the daily dose comprises at least 90 mg of the oxycodone or pharmaceutically acceptable salt thereof;

wherein the daily dose is administered as two or more prolonged release oral dosage forms.

In particular embodiments of the above methods of treating breakthrough pain, the patient is administered the immediate release oxycodone, without an opioid antagonist, for treating breakthrough pain in a dose per day that is equivalent to 10% to 120% of the oxycodone in the daily dose, such as equivalent to 5%, 10%, 12%, 15%, 17%, 20%, 25%, 30%, or 35% to 50%, 60%, 70%, 80%, 90%, 100%, 110%, or 120% of the oxycodone in the daily dose per day. For example, the patient may be administered the immediate release oxycodone in a dose per day that is equivalent to 10% to 120%, 15% to 100%, 17% to 80%, or 20 to 70%, such as about 20%, 25%, 30%, 40%, 50%, 60%, 70%, 75%, 80%, 90%, 100%, 110%, or 120% of the oxycodone in the daily dose. In certain embodiments, the patient is administered the immediate release oxycodone in a dose that is equivalent to ⅛, ⅙, ¼, ⅓, or ½ of the oxycodone in the daily dose. In some embodiments, the patient is administered the immediate release oxycodone once-a-day, or multiple times a day, such as 2, 3, 4, 5, 6, 7, or 8 times per day or up to 2, 3, 4, 5, 6, 7, or 8 times per day. In particular embodiments, the patient is administered the immediate release oxycodone in a dose, up to 6 times per day, that is equivalent to ⅙ of the oxycodone in the daily dose.

In certain embodiments, the present disclosure provides a method of normalizing bowel function in a patient on opioid therapy comprising orally administering to the patient a daily dose comprising oxycodone or a pharmaceutically acceptable salt thereof and naloxone or a pharmaceutically acceptable salt thereof in a 2:1 weight ratio;

wherein the daily dose comprises at least 90 mg of the oxycodone or pharmaceutically acceptable salt thereof; and

wherein the daily dose is administered as two or more prolonged release oral dosage forms.

In some embodiments, the present disclosure provides a method of treating pain and normalizing bowel function in a patient comprising orally administering to the patient a daily dose comprising oxycodone or a pharmaceutically acceptable salt thereof and naloxone or a pharmaceutically acceptable salt thereof in a 2:1 weight ratio;

wherein the daily dose comprises at least 90 mg of the oxycodone or pharmaceutically acceptable salt thereof; and

wherein the daily dose is administered as two or more prolonged release oral dosage forms.

In some instances of the methods described above, the patient is on opioid therapy prior to the method, for example from administration of the daily dose or administration of other opioid regimens. For example, the patient may have been on opioid therapy for a certain time period, such as at least 2, 4, 6, 8, 10, 12, 18, or 24 weeks or at least 1, 2, 3, 4, 6, or 8 months or at least 1, 1.5, 2, 2.5, or 3 years. In some embodiments, the opioid therapy treats chronic pain of the patient. In certain embodiments, administration of other opioid regimens include administration of oxycodone, morphine, levorphenol, meperdine, hydrocodone, codeine, dihydrocodeine, hydromorphone, propoxyphene, methadone, oxymorphone, buprenorphine, or pharmaceutically acceptable salts of any of these, or a combination thereof, particularly oxycodone or a pharmaceutically acceptable salt thereof.

In other embodiments, the patient is not on opioid therapy prior to the method, i.e., the patient is opioid naïve.

In particular embodiments, the patient treated by the methods described herein (e.g., by administration of the daily dose) is suffering from cancer. The patient may be suffering from pain derived from the cancer or derived from methods of treating the cancer (such as chemotherapy and/or radiotherapy) or both. In some embodiments, the present methods for treating pain (e.g., breakthrough or chronic pain) treat these types of pain associated with cancer. In certain embodiments, the present methods for improving bowel function, maintaining bowel function, or normalizing bowel function comprise administering the daily dose to the patient suffering from cancer.

In some embodiments, the present disclosure provides a method of treating pain in a patient suffering from cancer comprising orally administering to the patient a daily dose comprising oxycodone or a pharmaceutically acceptable salt thereof and naloxone or a pharmaceutically acceptable salt thereof in a 2:1 weight ratio;

wherein the daily dose comprises at least 90 mg of the oxycodone or pharmaceutically acceptable salt thereof; and

wherein the daily dose is administered as two or more prolonged release oral dosage forms.

In certain embodiments herein involving improving bowel function, the improvement in bowel function is relative to that experienced from administering a corresponding naloxone-free daily dose (e.g., relative to placebo). For example, the improvement in bowel function may be assessed by the difference in bowel function index (BFI) scores between administering the daily dose and administering a corresponding naloxone-free daily dose (e.g., each relative to baseline). In certain instances, the difference in BFI scores is at least 10, 11, 12, 13, 14, 15, 16, 17, 18, 19, or 20, particularly at least 12, 14, 16, or 18, more particularly at least 12. In some embodiments, the difference in BFI scores is from 12 to 21, 12 to 20, 12 to 19, 12 to 18, or 12 to 17 or from 14 to 21, 14 to 20, 14 to 19, 14 to 18, or 14 to 17. In some instances, the difference in BFI scores is at least 21, 22, 24, 26, 28, 30, 32, 33, or 34. In some embodiments, the difference in BFI scores is from 12 to 22, 14 to 22, 16 to 22, or 18 to 22 or from 12 to 21, 14 to 21, 16 to 21, or 18 to 21.

In particular embodiments herein involving maintaining or normalizing bowel function, the maintenance or normalization of bowel function is relative to that experienced by a patient not on opioid therapy, a patient who has not been administered an opioid, or a non-constipated patient (but may have pain, such as chronic pain); that is, the patient subsequent to treatment by the present method exhibits bowel function levels similar to that of a patient not on opioid therapy, a patient who has not been administered an opioid, or a non-constipated patient. For example, the maintenance or normalization of bowel function may be assessed by the difference in BFI scores for the patient on opioid therapy (or receiving the daily dose) and the patient not on opioid therapy or not constipated. In certain instances, the difference in BFI scores is equal to or less than 13, 12, 11, 10, 9, 8, 7, 6, 5, 4, or 3, particularly equal to or less than 12, 10, 8, or 6, more particularly less than 12. In some embodiments, the difference in BFI scores is from 12 to 20, 12 to 19, 12 to 18, or 12 to 17 or from 14 to 20, 14 to 19, 14 to 18, or 14 to 17.

In other embodiments, the maintenance or normalization of bowel function may be assessed by comparing the BFI score for the patient on opioid therapy (or receiving the daily dose) and known BFI reference scores for subjects not on opioid therapy or not constipated (see, e.g., Ueberall et al., J. Int. Med. Res. 39(1), 41-50 (2011), incorporated by reference herein). In certain instances, the BFI for the patient on opioid therapy (or receiving the daily dose) is less than 34, 32, 30, 29, 28, 27, 26, 25, 24, 23, 22, 21, or 20, such as less than 32, 30, 29, 27, or 25. In some embodiments, the BFI for the patient on opioid therapy (or receiving the daily dose) is from 20 to 32, 20 to 30, 20 to 29, 20 to 27, or 20 to 25.

In some embodiments, BFI scores are measured by taking the mean symptom scores of two or three symptoms associated with bowel function, such as defecation, feeling of incomplete bowel evacuation, and judgment of constipation.

In certain embodiments, improving bowel function or normalizing bowel function comprises reduction in the number or hardness or dryness of stools; reduction of straining, bloating, abdominal cramping, or abdominal distension associated with bowel movements; reduction of gastric reflux or incomplete bowel evacuation; and/or particularly reducing OIC.

In some embodiments, maintaining bowel function comprises reducing or preventing an increase in the number or hardness or dryness of stools; the amount of straining, bloating, abdominal cramping, or abdominal distension associated with bowel movements; the amount of gastric reflux or incomplete bowel evacuation; and/or particularly the level of OIC.

In certain embodiments, administration of the daily dose in the methods described herein provides analgesia to the patient.

In some embodiments, the methods described herein result in a patient BFI score equal to or less than 45, 42, 40, 39, 38, 37, 36, 35, 34, 33, 32, 31, 30, 29, 28, 27, 26, 25, 24, 23, 22, or 21, particularly equal to or less than 35, 33, 31, or 29. In some embodiments, the patient BFI score is from 21, 22, 23, 24, 25, 26, 27, or 28 to 33, 34, 35, 36, 37, 38, 39, 40, or 45, such as from 22 to 45, 22 to 42, 22 to 40, 22 to 38, or 22 to 35.

In certain embodiments, the daily dose comprises at least or greater than 90, 100, 110, 120, 130, 140, 150, 160, 170, 180, 190, 200, 220, or 240 mg of oxycodone or pharmaceutically acceptable salt thereof.

In some embodiments, the daily dose comprises from 90, 100, 110, 120, 130, 140, or 150 mg to 160, 170, 180, 190, 200, 210, 220, 230, or 240 mg oxycodone or a pharmaceutically acceptable salt thereof. In certain embodiments, the daily dose comprises 90 mg to 480 mg, 90 mg to 240 mg, 90 mg to 200 mg, 90 mg to 180 mg, or 90 mg to 160 mg oxycodone or a pharmaceutically acceptable salt thereof, such as 100 mg to 480 mg, 100 mg to 240 mg, 100 mg to 200 mg, 100 mg to 180 mg, or 100 mg to 160 mg oxycodone or a pharmaceutically acceptable salt thereof. In other embodiments, the daily dose comprises 130 mg to 480 mg, 130 mg to 240 mg, 130 mg to 200 mg, 130 mg to 180 mg, or 130 mg to 160 mg oxycodone or a pharmaceutically acceptable salt thereof.

In particular embodiments, the daily dose comprises 90, 100, 110, 120, 130, 140, 150, 160, 170, 180, 190, 200, 220, or 240 mg oxycodone or a pharmaceutically acceptable salt thereof, particularly 100, 120, 140, 160, or 180 mg oxycodone or a pharmaceutically acceptable salt thereof, in particular 120, 140, or 160 mg oxycodone or a pharmaceutically acceptable salt thereof.

The daily dose further comprises an amount of naloxone or pharmaceutically acceptable salt thereof that is derivable from the amount of oxycodone or pharmaceutically acceptable salt thereof in the daily dose because the oxycodone and the naloxone are in a 2:1 weight ratio, respectively.

In certain embodiments, the daily dose is administered as one prolonged release oral dosage form. In other embodiments, the daily dose is administered as multiple prolonged release oral dosage forms, i.e., two or more prolonged release oral dosage forms, such as 2, 3, 4, 5, 6, 7, or 8 prolonged release oral dosage forms, in particular as 2, 4, or 6 prolonged release oral dosage forms. In some embodiments, the prolonged release oral dosage forms have equal doses with respect to each other. In other embodiments, the prolonged release dosage forms have unequal doses with respect to each other.

In some embodiments, the daily dose is administered in two administrations at approximately (e.g., within 60, 45, 30, 15. or 5 minutes) 12-hour intervals. In certain embodiments, the two administrations comprise equal doses. In other embodiments, the two administrations comprise different doses.

In preferred embodiments, the prolonged release oral dosage form includes both the oxycodone or pharmaceutically acceptable salt thereof and the naloxone or pharmaceutically acceptable salt thereof. When the prolonged release oral dosage form includes both the oxycodone and the naloxone, as noted above, the daily dose may be administered as one prolonged release oral dosage form or as multiple prolonged release dosage forms (e.g., 1, 2, 3, or 4 dosage forms every 12 hours) to achieve the daily dose.

In other embodiments, the oxycodone or pharmaceutically acceptable salt thereof and the naloxone or pharmaceutically acceptable salt thereof are administered in separate prolonged release oral dosage forms. Such a scenario necessarily requires that the daily dose is achieved by administration of multiple prolonged release oral dosage forms, e.g., dosage form(s) that include the oxycodone and dosage form(s) that include the naloxone.

In some embodiments, the daily dose is administered as two prolonged release dosage forms, each administered at approximately 12-hour intervals. In other embodiments, the daily dose is administered as four prolonged release dosage forms, with two of the four prolonged release dosage forms administered at approximately 12-hour intervals. In further embodiments, the daily dose is administered as six prolonged release dosage forms, with three of the six prolonged release dosage forms administered at approximately 12-hour intervals. In other embodiments, the daily dose is administered as eight prolonged release dosage forms, with four of the eight prolonged release dosage forms administered at approximately 12-hour intervals.

In certain embodiments, the daily dose is administered as part of an on-going therapeutic regimen. For instance, in some embodiments, the daily dose is administered daily for a period of at least 2, 4, 5, 6, 7, 8, 10, 12, 14, 16, 18, 20, 24, 36, or 48 weeks, particularly, at least 4, 5, 8, or 24 weeks.

### Definitions

In describing the present invention, the following terms are used as indicated below.

As used herein, the singular forms “a”, “an”, and “the” include plural references unless the context clearly indicates otherwise.

The term “about” in the context of the present invention denotes an interval of accuracy that a person skilled in the art will understand to still ensure the technical effect of the feature in question. The term typically indicates a deviation from the indicated numerical value of ±10% and preferably ±5%.

The term “comprising” is not limiting. For the purposes of the present invention, the term “consisting of” is considered to be a preferred embodiment of the term “comprising”. If hereinafter a group is defined to comprise at least a certain number of embodiments, this is also meant to encompass a group which preferably consists of these embodiments only. The term “consisting essentially of” is also considered to be a preferred embodiment of the term “comprising”.

The term “daily dose” as used herein refers to the amount of oxycodone or pharmaceutically acceptable salt thereof and naloxone or pharmaceutically acceptable salt thereof, which are administered in combination within each 24-hour period according to the methods disclosed herein. If, for example, the daily dose is stated as comprising 90 mg to 200 mg of oxycodone or a pharmaceutically acceptable salt thereof, this means that 90 mg to 200 mg of the oxycodone or pharmaceutically acceptable salt thereof is administered (with the corresponding amount of naloxone or a pharmaceutically acceptable salt thereof) to the patient either in a single administration step or in smaller amounts in multiple administration steps resulting in total in the overall dose of 90 mg to 200 mg per day.

If reference is made to a specific daily dose comprising oxycodone or a pharmaceutically acceptable salt thereof, e.g. 160 mg, and the oxycodone is administered in a dosage form or in multiple dosage forms, which may further contain(s) the naloxone or pharmaceutically acceptable salt thereof, the corresponding daily dose of naloxone or pharmaceutically acceptable salt thereof will be 80 mg because the oxycodone and the naloxone are in a 2:1 ratio by weight, respectively. Such a daily dose maybe called a “160/80” dose (oxycodone or pharmaceutically acceptable salt thereof to naloxone or pharmaceutically acceptable salt thereof). If reference is made herein to a “naloxone-free daily dose”, this means that naloxone is not co-administered but that only oxycodone is administered in the given daily dose.

In the methods described herein, the weight ratio of the two actives oxycodone or a pharmaceutically acceptable salt thereof and naloxone or a pharmaceutically acceptable salt thereof is typically 2:1. In some embodiments, the weight ratio is calculated on the basis of the free bases of the two actives (i.e., oxycodone free base to naloxone free base). In other embodiments, the weight ratio is calculated on the basis of the salts of the two actives, in particular the hydrochloride salt of the two actives (i.e., oxycodone HCl to naloxone HCl). Unless stated otherwise, the weight ratio is calculated on the basis of anhydrous variants of the two actives (e.g., anhydrous oxycodone HCl to anhydrous naloxone HCl or anhydrous oxycodone free base to anhydrous naloxone free base). This does not mean that hydrates of one or both of the actives cannot be used herein (e.g., naloxone HCl dihydrate), only that the 2:1 weight ratio is based on the corresponding amount of anhydrous active(s). If the free bases are not comprised per se in the dosage form(s), but rather as e.g. the hydrochloride salts or any other salts, the amounts of the non-free base forms can routinely be derived from the corresponding amounts of the free bases and vice versa. Similarly, if anhydrous variants of the corresponding actives are not used per se in the dosage form(s), but rather hydrate(s), the amounts of the anhydrous form(s) can be routinely derived from the corresponding amounts of the hydrates and vice versa.

The term “bowel function” as used herein in particular but not exclusively refers to opioid-induced bowel dysfunction (OIBD), wherein OIBD comprises hard dry stools, straining, bloating, abdominal cramping, abdominal distension, increased gastric reflux, incomplete bowel evacuation and as the most distressing lead symptom opioid-induced constipation (OIC). The term “maintaining bowel function” refers to methods that maintain the patient's bowel function ability (e.g., as measured by BFI score) at levels described herein following the particular method. In contrast, the term “normalizing bowel function” refers to methods that improve the patient's bowel function ability (e.g., as measured by BFI score)—which may have become reduced through opioid therapy or a disease state (such as cancer or an intestinal disease)—to levels described herein following the particular method.

The “BFI” score or “bowel function index” score as used herein is described in detail e.g. in Rentz et al., J. Med. Econ., 12(0), 371-383 (2009), incorporated herein by reference. As disclosed in Rentz et al., 2009, a BFI score change of ≥12 points represents a clinically meaningful change, in particular for OIC.

The term “pain” means moderate to severe, acute and chronic pain of malignant and non-malignant origin, in particular severe to most severe, acute and chronic pain of malignant and non-malignant origin. In certain embodiments, methods of treating pain herein include methods of treating cancer pain, i.e., pain derived from a cancer or from a method of treating the cancer.

The term “patient” means a subject, particularly a human, who has presented a clinical manifestation of a particular symptom or symptoms suggesting the need for treatment, who is treated preventatively or prophylactically for a condition, or who has been diagnosed with a condition to be treated. The term “subject” is inclusive of the definition of the term “patient” and does not exclude individuals who are entirely normal in all respects or with respect to a particular condition.

Whenever reference is made herein to a “difference in bowel function index scores”, this means that this difference is not to be determined for a single patient, i.e. wherein the single patient is administered the two different administration regimens under comparison (in particular, one containing naloxone and one without naloxone), but instead the difference in BFI scores is determined between different patients. This can in particular be a patient treated with oxycodone only vs. a patient treated with the combination of oxycodone and naloxone, or results from previous studies carried out with patients treated with oxycodone only.

The above definition also applies to the situation of a patient treated with the combination of oxycodone and naloxone vs. a patient not treated with an opioid or not on opioid therapy (such as with e.g. oxycodone). Again, a difference is not to be determined for a single patient, i.e. the single patient is administered the two different administration regimens under comparison (in particular, a combination of oxycodone and naloxone and no opioid), but instead the difference in BFI scores is determined between different patients. This can in particular be a patient treated with the combination of oxycodone and naloxone vs. a patient not treated with an opioid, or results from previous studies carried out with patients not treated with opioids or not on opioid therapy.

“Pharmaceutically acceptable salts” include, but are not limited to, inorganic acid salts such as hydrochloride, hydrobromide, sulfate, bisulfate, nitrate, phosphate and the like; organic acid salts such as myristate, formate, acetate, trifluoroacetate, maleate, malate, fumarate, succinate, tartrate, bitartrate, and the like; sulfonates such as methanesulfonate, benzenesulfonate, p-toluenesulfonate and the like; amino acid salts such as arginate, asparaginate, glutamate and the like; metal salts such as sodium salt, potassium salt, cesium salt and the like; alkaline earth metals such as calcium salt, magnesium salt and the like; and organic amine salts such as triethylamine salt, pyridine salt, picoline salt, ethanolamine salt, triethanolamine salt, discyclohexylamine salt, N,N′-dibenzylethylenediamine salt and the like.

A particularly preferred salt for both actives is the hydrochloride salt. When the hydrochloride salt of naloxone is used, it is even more preferred to use naloxone hydrochloride dihydrate.

The oxycodone base and pharmaceutically acceptable salts thereof may be present in solvent free form such as in the anhydrous form, and as solvates such as the hydrates, and as complexes, and mixtures thereof.

The naloxone base and pharmaceutically acceptable salts thereof may be present in solvent free form such as in the anhydrous form, and as solvates such as the hydrates, and as complexes, and mixtures thereof.

PCT International Publication WO 2005/097801, U.S. Pat. No. 7,129,248, and U.S. Patent Application Publication 2006/0173029, all of which are hereby incorporated by reference, describe processes for preparing oxycodone hydrochloride having a 14-hydroxycodeinone level of less than about 25 ppm, preferably of less than about 15 ppm, less than about 10 ppm, or less than about 5 ppm, more preferably of less than about 2 ppm, less than about 1 ppm, less than about 0.5 ppm or less than about 0.25 ppm.

The term “ppm” as used herein means “parts per million”. Regarding 14-hydroxycodeinone, “ppm” means parts per million of 14-hydroxycodeinone in a particular sample product. The 14-hydroxycodeinone level can be determined by any method known in the art, preferably by HPLC analysis using UV detection.

In certain embodiments of the present invention, wherein the active agent is oxycodone hydrochloride, oxycodone hydrochloride is used having a 14-hydroxycodeinone level of less than about 25 ppm, preferably of less than about 15 ppm, less than about 10 ppm, or less than about 5 ppm, more preferably of less than about 2 ppm, less than about 1 ppm, less than about 0.5 ppm or less than about 0.25 ppm.

### The Prolonged Release Oral Dosage Form of the Present Invention

### Amounts of the Actives in a Dosage Form According to the Invention

It is preferred that a prolonged release oral dosage form of the present invention comprises both actives, i.e. oxycodone or a pharmaceutically acceptable salt thereof and naloxone or a pharmaceutically acceptable salt thereof. Generally, the dosage form can comprise oxycodone or a pharmaceutically acceptable salt thereof in an amount range of about 1 mg to about 240 mg, such as about 20, 30, 40, 50, 60, 70, 80, 90, 100, 110, 120, or 130 mg to about 140, 150, 160, 180, 200, 220, or 240 mg. In certain embodiments, the dosage form comprises oxycodone or a pharmaceutically acceptable salt thereof in an amount range of about 40 mg to about 240 mg, about 50 mg to about 240 mg, about 60 mg to about 240 mg, about 80 mg to about 240 mg, about 90 mg to about 240 mg, about 120 mg to about 240 mg, or about 130 mg to about 240 mg. In some embodiments, the dosage form comprises oxycodone or a pharmaceutically acceptable salt thereof in an amount range of about 40 mg to about 160 mg, about 50 mg to about 160 mg, about 60 mg to about 160 mg, about 80 mg to about 160 mg, about 90 mg to about 160 mg, about 120 mg to about 160 mg, or about 130 mg to about 160 mg.

### Particular Amounts of the Actives in a Dosage Form of the Invention

In certain embodiments, the dosage forms comprise amounts of equivalent to about 2.5 mg oxycodone HCl and about 1.25 mg naloxone HCl; about 5 mg oxycodone HCl and about 2.5 mg naloxone HCl; about 10 mg oxycodone HCl and about 5 mg naloxone HCl; about 20 mg oxycodone HCl and about 10 mg naloxone HCl; about 40 mg oxycodone HCl and about 20 mg naloxone HCl; about 80 mg oxycodone HCl and about 40 mg naloxone HCl; and about 100 mg oxycodone HCl and about 50 mg naloxone HCl. Even more preferred are amounts of 40 mg oxycodone HCl and 20 mg naloxone HCl; 60 mg oxycodone HCl and 30 mg naloxone HCl; 80 mg oxycodone HCl and 40 mg naloxone HCl; 100 mg oxycodone HCl and 50 mg naloxone HCl; 120 mg oxycodone HCl and 60 mg naloxone HCl; 140 mg oxycodone HCl and 70 mg naloxone HCl; 160 mg oxycodone HCl and 80 mg naloxone HCl; and 180 mg oxycodone HCl and 90 mg naloxone HCl.

### Particular Daily Doses of the Invention

In some embodiments, the present methods described herein comprise administering a daily dose comprising oxycodone or a pharmaceutically acceptable salt thereof and naloxone or a pharmaceutically acceptable salt thereof in a 2:1 weight ratio, wherein the daily dose comprises 90 mg of the oxycodone or pharmaceutically acceptable salt thereof, and the daily dose is administered as a 40/20 dose and a 5/2.5 dose twice-a-day, or two 20/10 doses and a 5/2.5 dose twice-a-day, or a 30/15 dose and a 15/7.5 dose twice-a-day, such as approximately every 12 hours.

In certain embodiments, the present methods described herein comprise administering a daily dose comprising oxycodone or a pharmaceutically acceptable salt thereof and naloxone or a pharmaceutically acceptable salt thereof in a 2:1 weight ratio, wherein the daily dose comprises 100 mg of the oxycodone or pharmaceutically acceptable salt thereof, and the daily dose is administered as a 30/15 dose and a 20/10 dose twice-a-day, such as approximately every 12 hours.

In some embodiments, the present methods described herein comprise administering a daily dose comprising oxycodone or a pharmaceutically acceptable salt thereof and naloxone or a pharmaceutically acceptable salt thereof in a 2:1 weight ratio, wherein the daily dose comprises 120 mg of the oxycodone or pharmaceutically acceptable salt thereof, and the daily dose is administered as a 60/30 dose twice-a-day, or two 30/15 doses twice-a-day, such as approximately every 12 hours.

In certain embodiments, the present methods described herein comprise administering a daily dose comprising oxycodone or a pharmaceutically acceptable salt thereof and naloxone or a pharmaceutically acceptable salt thereof in a 2:1 weight ratio, wherein the daily dose comprises 140 mg of the oxycodone or pharmaceutically acceptable salt thereof, and the daily dose is administered as a 40/20 dose and a 30/15 dose twice-a-day, or a 60/30 dose and a 10/5 dose twice-a-day, such as approximately every 12 hours.

In some embodiments, the present methods described herein comprise administering a daily dose comprising oxycodone or a pharmaceutically acceptable salt thereof and naloxone or a pharmaceutically acceptable salt thereof in a 2:1 weight ratio, wherein the daily dose comprises 160 mg of the oxycodone or pharmaceutically acceptable salt thereof, and the daily dose is administered as a 80/40 dose twice-a-day, or two 40/20 doses twice-a-day, such as approximately every 12 hours.

In certain embodiments, the present methods described herein comprise administering a daily dose comprising oxycodone or a pharmaceutically acceptable salt thereof and naloxone or a pharmaceutically acceptable salt thereof in a 2:1 weight ratio, wherein the daily dose comprises 180 mg of the oxycodone or pharmaceutically acceptable salt thereof, and the daily dose is administered as a 60/30 dose and a 30/15 dose twice-a-day, or a 80/10 dose and a 10/5 dose twice-a-day, such as approximately every 12 hours.

In some embodiments, the present methods described herein comprise administering a daily dose comprising oxycodone or a pharmaceutically acceptable salt thereof and naloxone or a pharmaceutically acceptable salt thereof in a 2:1 weight ratio, wherein the daily dose comprises 200 mg of the oxycodone or pharmaceutically acceptable salt thereof, and the daily dose is administered as a 60/30 dose and a 40/20 dose twice-a-day, such as approximately every 12 hours.

In certain embodiments, the present methods described herein comprise administering a daily dose comprising oxycodone or a pharmaceutically acceptable salt thereof and naloxone or a pharmaceutically acceptable salt thereof in a 2:1 weight ratio, wherein the daily dose comprises 220 mg of the oxycodone or pharmaceutically acceptable salt thereof, and the daily dose is administered as a 80/40 dose and a 30/15 dose twice-a-day, such as approximately every 12 hours.

In some embodiments, the present methods described herein comprise administering a daily dose comprising oxycodone or a pharmaceutically acceptable salt thereof and naloxone or a pharmaceutically acceptable salt thereof in a 2:1 weight ratio, wherein the daily dose comprises 240 mg of the oxycodone or pharmaceutically acceptable salt thereof, and the daily dose is administered as a 80/40 dose and a 40/20 dose twice-a-day, or two 60/30 doses twice-a-day, such as approximately every 12 hours.

Release Rates of the Two Actives from a Dosage Form of the Present Invention

It is preferred that a dosage form comprising oxycodone or a pharmaceutically acceptable salt thereof and naloxone or a pharmaceutically acceptable salts thereof releases in vitro, when measured using the Ph. Eur. Paddle Method at 50 rpm in 0.1 N hydrochloric acid, pH 1.2 at 37° C. and using UV detection at 230 nm, about 5% to about 40% of oxycodone or a pharmaceutically acceptable salt thereof by weight and about 5% to about 40% of naloxone or a pharmaceutically acceptable salt thereof by weight at 15 min; about 20% to about 50% of oxycodone or a pharmaceutically acceptable salt thereof by weight and about 20% to about 50% of naloxone or a pharmaceutically acceptable salt thereof by weight at 1 hour; about 30% to about 60% of oxycodone or a pharmaceutically acceptable salt thereof by weight and about 30% to about 60% of naloxone or a pharmaceutically acceptable salt thereof by weight at 2 hours; about 50% to about 80% of oxycodone or a pharmaceutically acceptable salt thereof by weight and about 50% to about 80% of naloxone or a pharmaceutically acceptable salt thereof by weight at 4 hours; about 70% to about 95% of oxycodone or a pharmaceutically acceptable salt thereof by weight and about 70% to about 95% of naloxone or a pharmaceutically acceptable salt thereof by weight at 7 hours; and/or more than about 80% of oxycodone or a pharmaceutically acceptable salt thereof by weight and more than about 80% of naloxone or a pharmaceutically acceptable salt thereof by weight at 10 hours.

In a particularly preferred embodiment relating to the in vitro release of a prolonged release dosage form comprising oxycodone or a pharmaceutically acceptable salt thereof and naloxone or a pharmaceutically acceptable salts thereof, said dosage form releases in vitro, when measured using the Ph. Eur. Paddle Method at 50 rpm in 0.1 N hydrochloric acid, pH 1.2 at 37° C. and using UV detection at 230 nm, about 10% to about 30% of oxycodone or a pharmaceutically acceptable salt thereof by weight and about 10% to about 30% of naloxone or a pharmaceutically acceptable salt thereof by weight at 15 min; about 30% to about 45% of oxycodone or a pharmaceutically acceptable salt thereof by weight and about 30% to about 45% of naloxone or a pharmaceutically acceptable salt thereof by weight at 1 hour; about 40% to about 60% of oxycodone or a pharmaceutically acceptable salt thereof by weight and about 40% to about 60% of naloxone or a pharmaceutically acceptable salt thereof by weight at 2 hours; about 55% to about 70% of oxycodone or a pharmaceutically acceptable salt thereof by weight and about 55% to about 75% of naloxone or a pharmaceutically acceptable salt thereof by weight at 4 hours; about 75% to about 90% of oxycodone or a pharmaceutically acceptable salt thereof by weight and about 75% to about 90% of naloxone or a pharmaceutically acceptable salt thereof by weight at 7 hours; and/or more than about 85% of oxycodone or a pharmaceutically acceptable salt thereof by weight and more than about 85% of naloxone or a pharmaceutically acceptable salt thereof by weight at 10 hours.

Further, in certain embodiments a prolonged release dosage form according to the invention releases the oxycodone or a pharmaceutically acceptable salt thereof and the naloxone or a pharmaceutically acceptable salt thereof at substantially equal release rates, such as with release rates that differ by 25% or less, 20% or less, 15% or less, 10% or less, or 5% or less, for example, when measured using the Ph. Eur. Paddle Method at 50 rpm in 0.1 N hydrochloric acid, pH 1.2 at 37° C. and using UV detection at 230 nm.

The dosage form according to the present invention is preferably administered on a twice-a-day basis and thus provides its effect in vivo preferably for about 12 hours. However, it is generally also possible to adapt the release rate such that a once-a-day basis for the administration is achieved.

### The Prolonged Release Dosage Form of the Present Invention

In general, all dosage forms providing a prolonged release of the two actives may be used. Of course, a prolonged release dosage form capable of providing the afore-mentioned in vitro release rates is the most preferred prolonged release dosage form.

In a preferred embodiment, the prolonged release dosage form comprises a prolonged release matrix in order to achieve the prolonged release. In an alternative preferred embodiment, the prolonged release dosage form comprises a prolonged release coating in order to achieve the prolonged release of the active agents. In a further alternative preferred embodiment, the prolonged release dosage form is an osmotic prolonged release dosage form. When a prolonged release matrix dosage form is used, the matrix preferably comprises a fatty alcohol and/or a hydrophobic polymer such as an alkylcellulose with ethylcellulose being particularly preferred. Other structural components of the different prolonged release dosage forms are described below.

### Further Features of the Dosage Form of the Present Invention

The dosage form of the present invention may comprise further pharmaceutically acceptable ingredients and/or adjuvants, such as e.g. lubricants, fillers, binders, flowing agents, colorants, flavorants, surfactants, pH-adjusters, anti-tacking agents and/or combinations thereof.

Preferably, the dosage form is selected from the group consisting of a tablet, a capsule, a multiparticulate, a dragée, a granulate, a liquid and a powder. A particularly preferred dosage form is a tablet or a multi-particulate.

The dosage form according to the invention may comprise at least one further pharmaceutically active agent providing a further desired pharmaceutical effect in addition to the two active agents, i.e. oxycodone or a pharmaceutically acceptable salt thereof and naloxone or a pharmaceutically acceptable salt thereof. However, it is preferred that the dosage form according to the invention comprises the two actives oxycodone or a pharmaceutically acceptable salt thereof and naloxone or a pharmaceutically acceptable salt thereof as the sole pharmaceutically active agents.

### A Particular Dosage Form According to the Present Invention

It can be preferred that the dosage form is an oral prolonged release dosage form comprising oxycodone or a pharmaceutically acceptable salt thereof and naloxone or a pharmaceutically acceptable salt thereof, wherein the two actives are present in a weight ratio of 2:1 with amounts of oxycodone of equivalent to 40, 60, or 80 mg oxycodone HCl (particularly 80 mg oxycodone HCl) and of naloxone of equivalent to 20, 30, or 40 mg naloxone HCl (particularly 40 mg naloxone HCl), wherein the dosage form releases in vitro, when measured using the Ph. Eur. Paddle Method at 50 rpm in 0.1 N hydrochloric acid, pH 1.2 at 37° C. and using UV detection at 230 nm, about 5% to about 40% of oxycodone or a pharmaceutically acceptable salt thereof by weight and about 5% to about 40% of naloxone or a pharmaceutically acceptable salt thereof by weight at 15 min; about 20% to about 50% of oxycodone or a pharmaceutically acceptable salt thereof by weight and about 20% to about 50% of naloxone or a pharmaceutically acceptable salt thereof by weight at 1 hour; about 30% to about 60% of oxycodone or a pharmaceutically acceptable salt thereof by weight and about 30% to about 60% of naloxone or a pharmaceutically acceptable salt thereof by weight at 2 hours; about 50% to about 80% of oxycodone or a pharmaceutically acceptable salt thereof by weight and about 50% to about 80% of naloxone or a pharmaceutically acceptable salt thereof by weight at 4 hours; about 70% to about 95% of oxycodone or a pharmaceutically acceptable salt thereof by weight and about 70% to about 95% of naloxone or a pharmaceutically acceptable salt thereof by weight at 7 hours; and/or more than about 80% of oxycodone or a pharmaceutically acceptable salt thereof by weight and more than about 80% of naloxone or a pharmaceutically acceptable salt thereof by weight at 10 hours.

In the dosage form employed herein, it is furthermore preferred that the dosage form comprises oxycodone hydrochloride and naloxone hydrochloride dihydrate. Although all prolonged release dosage forms may be used, it is further most preferred that the dosage form in its most preferred embodiment comprises a prolonged release matrix.

### The Release Behaviour of the Oral Dosage Form of the Present Invention

In contrast to an “immediate release”, a “prolonged release” dosage form in accordance with the present invention refers to pharmaceutical compositions which release in vitro ≤75% (by weight) of the pharmaceutically active agents, namely oxycodone and naloxone, at 45 min.

In the context of the present invention, the term “immediate release” refers to pharmaceutical compositions showing a release of the active substances which is not deliberately modified by a special formulation design and/or manufacturing methods. For oral dosage forms this means that the dissolution profile of the active substances depends essentially on their intrinsic properties. Typically, the term “immediate release” refers to pharmaceutical compositions which release in vitro >75% (by weight) of the pharmaceutically active agents at 45 min.

Prolonged release properties may be obtained by different means such as by a coating which is then designated as a prolonged release coating, a matrix which is then designated as a prolonged release matrix or e.g. by an osmotic structure of the pharmaceutical composition.

In order to obtain “prolonged release” properties, one typically uses materials which are known to prolong the release from a dosage form comprising e.g. a prolonged release matrix and/or prolonged release coating. Typical examples are set out further below. The nature of the “prolonged release material” may depend on whether the release properties are attained by a “prolonged release matrix” or a “prolonged release coating”. The term “prolonged release materials” thus describes both types of materials. The term “prolonged release matrix material” indicates that a material is used for obtaining a prolonged release matrix. Likewise, the term “prolonged release coating material” indicate that a material is used for obtaining a prolonged release coating.

The term “prolonged release matrix formulation” refers to a pharmaceutical composition including at least one prolonged release material, and at least oxycodone and naloxone as the two pharmaceutically active agents. In a “prolonged release matrix formulation”, the “prolonged release materials” are combined with the pharmaceutically active agents to form a mixture from which the pharmaceutically active agents are released over prolonged periods of time, such as e.g. 8, 10, 12, 14, 16, 18, 20, 22 or 24 hours.

It is to be understood that a material will be considered to act as prolonged release material if the dissolution profile of the pharmaceutically active agents is slowed down compared to an immediate or conventional release formulation. If a prolonged release material can be used for manufacturing a prolonged release matrix, it will be considered as a prolonged release matrix material.

Pharmaceutically acceptable excipients which are used to adjust an already prolonged release to a specific profile are not necessarily considered to be prolonged release materials.

It is to be understood that a prolonged release matrix does not necessarily consist only of the pharmaceutically active agents and the prolonged release material. The prolonged release matrix may comprise in addition pharmaceutically acceptable excipients such as fillers, lubricants, glidants, etc. Examples of such excipients are set out below.

The term “prolonged release coating formulation” refers to a pharmaceutical composition including at least one prolonged release material, and oxycodone and naloxone as the two pharmaceutically active agents. In a “prolonged release coating formulation”, the “prolonged release materials” are disposed on the pharmaceutically active agents to form a diffusion barrier. Other than in prolonged release matrix formulation, the actives are not intimately mixed with the prolonged release material and the prolonged release coating does not form a three dimensional structure within which the actives are distributed. As the term implies, the prolonged release material forms a layer above the actives. The pharmaceutically active agents are released from a prolonged release coating formulation over prolonged periods of time, such as e.g. 8, 10, 12, 14, 16, 18, 20, 22 or 24 hours.

It is to be understood that a material will be considered to act as prolonged release material if the dissolution profile of the pharmaceutically active agents is slowed down compared to an immediate or conventional release formulation. If a prolonged release material can be used for manufacturing a prolonged release coating, it will be considered as a prolonged release coating material.

Pharmaceutically acceptable excipients which are used to adjust an already prolonged release to a specific profile are not necessarily considered to be prolonged release materials.

When it is mentioned that a prolonged release coating is disposed on pharmaceutically active agents, this is not to be construed as meaning that such a coating will necessarily be directly layered on such active pharmaceutically agents. Of course, if the pharmaceutically active agents, oxycodone and naloxone, are layered on a carries such as Nu-Pareil beads, the coating may be disposed directly thereon. However, the pharmaceutically active agents may also be first embedded in a polymer layer or e.g. a prolonged release matrix. Subsequently the prolonged release coating may be disposed on e.g. granules which comprise a prolonged release matrix or on tablets which are made from such granules by compression for example.

A pharmaceutical composition with a prolonged release coating may be obtained by combining the pharmaceutically active agents with a carries such as Nu-Pareil beads and disposing a prolonged release coating on said combinations. Such coating may be made from polymers such cellulose ethers with ethyl cellulose being preferred, acrylic resins, other polymers and mixtures thereof. Such prolonged release coatings may comprise additional excipients such as pore-formers, binders and the like.

It is further to be understood, that the term “prolonged release matrix formulation” does not exclude pharmaceutical compositions with a prolonged release matrix and an additional prolonged release coating being disposed on the matrix. Likewise the term “prolonged release coating formulation” does not exclude pharmaceutical compositions with a prolonged release coating which is disposed on prolonged release matrix.

The term “prolonged release dosage form” refers to the administration form of a pharmaceutical composition of the present invention comprising the two pharmaceutically active agents, i.e. oxycodone and naloxone, in prolonged release form as e.g. in form of a “prolonged release matrix formulation”, in the form of a “prolonged release coating formulation”, combinations thereof or in other prolonged release formulations such as osmotic formulations. The terms “prolonged release matrix formulation” and “prolonged release dosage form” can be used interchangeably if the prolonged release dosage form consists essentially of the prolonged release matrix formulation. This means that a prolonged release dosage form can comprise in addition to the prolonged release matrix e.g. cosmetic coatings and pharmaceutically acceptable excipients such fillers, lubricants, etc.

For some embodiments, the term “prolonged release matrix dosage form” may indicate that the dosage form comprises a prolonged release matrix as the sole structure being responsible for prolonging the release. This, however, does not exclude that the dosage form may comprise an immediate release portion.

For some embodiments, the term “prolonged release coating dosage form” may indicate that the dosage form comprises a prolonged release coating as the sole structure being responsible for prolonging the release. This, however, does not exclude that the dosage form may comprise an immediate release portion.

The release rates indicated always refer to the formulation such as a monolithic tablet or multi-particulates. The release rates will be chosen such that a pharmaceutical composition can be administered e.g. on a twice a day or once a day basis, i.e. every 12 hours or every 24 hours. Typically, the release will occur by diffusion through the prolonged release matrix and/or coating, erosion of the prolonged matrix and/or coating or combinations thereof.

The term “substantially equal release rate” as used herein means that the two active agents, i.e. oxycodone and naloxone, are released from the dosage form such that their % of release does not deviate by more than about 20%, preferably by not more than about 15% and most preferably by not more that about 10%. In the most preferred embodiment, i.e. in the about 10% range, this means for example for a prolonged release dosage form comprising oxycodone and naloxone that if about 20% of oxycodone or a pharmaceutically acceptable salt are released from the dosage form in vitro after 15 minutes, naloxone will be released within a range of about 10% to about 30%, most preferably also at about 20% at 15 minutes.

### Release Materials

The following description of suitable materials is to be understood as being not limiting. Rather, the release material may be any material that is known to be capable of imparting prolonged release properties on the active agents, oxycodone and naloxone, when being formulated into a dosage form.

### Prolonged Release Matrix Materials

Suitable materials for inclusion in a prolonged release matrix in order to provide a prolonged release matrix dosage form comprising oxycodone and naloxone include:
(a) Hydrophilic or hydrophobic polymers, such as gums, cellulose ethers, acrylic resins and protein derived materials. Of these polymers, the cellulose ethers, especially alkylcelluloses are preferred. The dosage form may conveniently contain between 1% and 80% (by weight) of one or more hydrophilic or hydrophobic polymers.
(b) Substituted or unsubstituted hydrocarbons, such as fatty acids, fatty alcohols, glycerol esters of fatty acids, oils, and waxes. Hydrocarbons having a melting point of between 25 and 90° C. are preferred. The hydrocarbons may be long chain (C₈-C₅₀, preferably C₁₂-C₄₀) hydrocarbons. The hydrocarbons may be digestible. The oils and waxes may be vegetable, animal, mineral or synthetic oils and waxes. Of these hydrocarbon materials, fatty (aliphatic) alcohols are preferred. The dosage form may conveniently contain up to 60% (by weight) of at least one digestible, long chain hydrocarbon.
(c) Polyalkylene glycols. The dosage form may suitably contain up to 60% (by weight) of one or more polyalkylene glycols.

In a preferred embodiment, the pharmaceutical dosage forms as described in the present invention will use a diffusion matrix for achieving prolonged release of oxycodone and naloxone from the pharmaceutical dosage form.

To this end, the diffusion matrix may be made from a hydrophobic polymer and/or a C₁₂-C₃₆ fatty alcohol.

As regards the hydrophobic polymer, use of a hydrophobic cellulose ether and particularly ethyl cellulose may be preferred.

As regards the fatty alcohol, use of lauryl, myristyl, stearyl, cetylstearyl, ceryl and/or cetylalcohol will be preferably considered. The use of stearyl alcohol is particularly preferred.

A particularly preferred embodiment relates to pharmaceutical dosage forms in which the prolonged release properties of oxycodone and naloxone are provided by a diffusion matrix which is made from a hydrophobic polymer such as from ethyl cellulose and a fatty alcohol. The matrices of some of the preferred embodiments of the invention, which may e.g. be made from the aforementioned combination of ethyl cellulose and stearyl alcohol, will be a substantially non-swellable diffusion matrix.

The term “substantially non-swellable diffusion matrix” indicates that the matrix will be substantially non-erosive, i.e. that the size of the matrix will not significantly increase upon contact with fluids. Typically, the volume of a substantially non-swellable diffusion matrix will increase at maximum up to 100%, preferably at maximum up to 75%, more preferably at maximum up to 50%, even more preferably at maximum up to 25% and most preferably at maximum up to 10% or at maximum up to 5% in volume upon contacting an aqueous solution.

Pharmaceutical dosage forms which comprise a hydrophobic polymer with hydrophobic cellulose ethers such as ethyl cellulose being preferred as the sole or one of the components for providing a prolonged release (non-swellable) diffusion matrix, will use an amount of such polymer of between 5 to 20%, preferably of between 6 and 15% by weight and more preferably of between 7 to 10% by weight. The percentages indicate the amount of the matrix-forming material with respect to the total weight of the pharmaceutical dosage form.

Pharmaceutical dosage forms, which comprise a fatty alcohol as the sole or one of the components for providing a prolonged release diffusion matrix, will use an amount of fatty alcohol in the matrix of between 10 to 40%, preferably of between 15 to 35% and more preferably of between 17 to 25% by weight. These percentages again indicate the amount of fatty alcohol based on the total weight of the dosage form.

The person skilled in the art is further aware that such a prolonged release matrix may also contain other pharmaceutically acceptable ingredients and excipients which are conventional in the pharmaceutical art such as lubricants, fillers, binders, flowing agents, colorants, flavorants, surfactants, pH-adjusters, anti-tacking agents and granulating aids. These excipients will typically have no substantial impact on the overall release behavior of the pharmaceutical dosage form.

Typical examples of fillers (diluents) comprise lactose, preferably anhydrous lactose, glucose, saccharose, starch and their hydrolysates, microcrystalline cellulose, cellatose, sugar alcohols such as sorbitol or mannitol, calcium salts like calcium hydrogen phosphate, dicalcium- or tricalcium phosphate. Granulating aids comprise inter alia povidone. Flowing agents and lubricants comprise inter alia highly dispersed silica, talcum, magnesium oxide, calcium stearate, magnesium stearate, sodium stearyl fumarate, fast like hydrated castor oil and glyceryl dibehenate. Binders can include hyproxypropylmethyl cellulose (hypromellose), hydroxypropyl cellulose, hydroxyethyl cellulose, polyvinyl pyrollidone (povidone), acetic acid vinyl ester (copovidone) and carboxymethycellulose sodium. Anti-tacking agents may include glycerol monostearate. Furthermore, a matrix-based dosage form may e.g. comprise a cosmetic coating.

### Prolonged Release Coating Materials

As mentioned above, prolonged release characteristics of a pharmaceutical dosage form may also be achieved by a film coating that governs the release of the active agents from the dosage form. To this end, the pharmaceutical dosage form may comprise a carrier, which is associated with the opioid agonist and the opioid antagonist. For example, one may use nonpareil beads, sugar beads etc. on and/or into which the pharmaceutically active agents are disposed.

Such active-associated carriers may then be overcoated with a coating that provides prolonged release characteristics. Suitable prolonged release coating materials include hydrophobic polymers such as cellulose ethers and/or acrylic polymer resins. Ethylcellulose may be preferred.

The prolonged release coatings may comprise other components such as hydrophilic substances including hydrophilic polymers such hydroxypropylmethylcellulose (HPMC), polyethylenglycols etc. These components may be used to adjust the prolonged release characteristics of the coatings. In case of e.g. HPMC, the substances may act as pore formers. The coating may, of course, also comprise additional pharmaceutically acceptable excipients, e.g. as set out above for the matrices.

### EXAMPLES

The present invention is now more fully described with reference to the accompanying examples. It should be understood, however, that the following description is illustrative only and should not be taken in any way as a restriction of the invention.

### Example 1

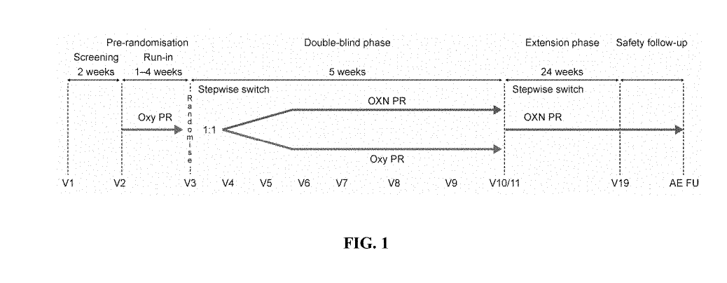

Example 1 was a multicentre, multiple-dose, randomised, double-blind, double-dummy, active-controlled, parallel-group study in male and female subjects with non-malignant or malignant pain requiring opioids to assess analgesic efficacy and symptoms of constipation secondary to opioid treatment. The study was comprised of three phases: a pre-randomization phase consisting of a screening period and a run-in period, a double-blind phase and an extension phase (FIG. 1). Eligible patients selected during the screening phase entered the Run-in phase, during which OxyPR was titrated to analgesic effect in order to determine the starting dose to be used after randomization. At Visit 2 (V2), opioid therapy was converted to open-label OxyPR and titrated to an effective analgesic daily dose of OxyPR between 100-160 mg (50, 60, 70 or 80 mg twice daily). Patients were provided with immediate-release oxycodone (Oxy IR) for breakthrough pain to be used up to six times per day at a dose of approximately ⅙ that of the total daily study medication, and oral bisacodyl 10 mg/day as laxative rescue medication. Patients were also given a daily diary to record analgesic rescue medication use, laxative rescue medication use, bowel function measures, and average pain over last 24 hours. These patients had to be on a stable dose of OxyPR twice daily for at least 4 consecutive days prior to randomization and have a pain score of ≤4 with no more than two doses of Oxy IR analgesic rescue medication per day for either the last 3 consecutive days or 4 of the last 7 days. Patients were randomly assigned in a 1:1 ratio to OXN PR or OxyPR for up to 5 weeks. The starting dose during the double-blind phase was dependent on the effective, stable analgesic dose established in the run-in period, but titration up to maximum daily dose of OXN PR 160/80 mg was permitted after 1 week.

There were two primary endpoints, the first of which was to demonstrate that patients taking OXN PR had improvement in symptoms of constipation as measured by the Bowel Function Index (BFI) compared with patients taking OxyPR tablets alone. The second was to demonstrate non-inferiority of OXN PR compared with OxyPR with respect to the analgesic efficacy based on average pain over last 24 hours as measured by the Pain Intensity Scale. Secondary endpoints were analgesic and laxative rescue medication, complete spontaneous bowel movements (CSBMs) and quality of life (EuroQol EQ-5D).

Enrolled patients were males and females aged ≥18 years with cancer and non-cancer pain requiring opioids according to World Health Organization (WHO) step III criteria and suffering from opioid-induced constipation caused or aggravated by opioids. Included patients were dissatisfied with their current analgesic medication due to lack of efficacy or unacceptable tolerability. The need for analgesic medication was defined as a documented history of requiring around-the-clock opioid therapy (100-160 mg OxyPR per day) for at least 5 weeks. Constipation caused or aggravated by opioids was confirmed by the patient and the investigator as an effect of the patient's pre-study opioid medication (at a comparable dose) and evidenced by a medical need of regular intake of laxatives to have at least three bowel evacuations per week or by having less than three bowel evacuations when not taking a laxative. Non-analgesic concomitant medications, including those medications for the treatment of depression, and the non-opioid analgesic medication dose were required to be stable at screening and to have remained stable throughout the double-blind phase of the study, as judged by the investigator. Exclusion criteria included: history of hypersensitivity to oxycodone, naloxone, related products or other ingredients of the study medications; any contraindication to bisacodyl or any components of the study medications; active alcohol or drug abuse and/or history of opioid abuse; unreported illicit drug use (including cannabis); any condition in which opioids are contraindicated. Patients were also excluded if they had taken naloxone ≤30 days prior to the start of the screening period or at screening; if they were taking or had taken monoamine oxidase inhibitors ≤2 weeks prior to the start of the screening period; or if they were suffering from diarrhea.

Patients were assessed at weeks 1, 2, 4 and 5 during the double-blind phase (at study site/home). Opioid-induced constipation was assessed using the BFI, daily laxative rescue medication use, and the number of CSBMs per week. The BFI score was the mean of the following three items subjectively assessed by the patient at each clinic visit: ease of defecation (numerical analogue scale (NAS), 0=easy/no difficulty; 100=severe difficulty), feeling of incomplete bowel evacuation (NAS, 0=not at all, 100=very strong), and judgment of constipation (NAS, 0=not at all, 100=very strong). Analgesic efficacy was measured as average pain over the last 24 hours based on the Pain Intensity Scale (a numerical rating scale 0-10 in which 0=no pain and 10=worst imaginable pain) assessed at each visit and in patient daily diaries, together with analgesic rescue medication use. Quality of life (QoL) was evaluated using the EuroQol EQ-5D-3L instrument. Adverse events were monitored throughout the 5-week study.

For the primary analysis of BFI endpoint, a mixed-model repeated measures (MMRM) analysis of covariance of the BFI scores was carried out with no missing data imputation. The repeated measures analysis included fixed-effect terms for treatment and time, random effect for centre or site, and pre-randomization value at the end of the Baseline Period. The same statistical MMRM model as used for the BFI was applied to analyse the Pain Intensity Scale results for average pain over last 24 hours at each visit. The superiority analysis on the BFI was performed using the FA population and the non-inferiority test on the Pain Intensity Scale was performed using the PP population. Superiority hypothesis tests applied a 5% two-sided significance level, while non-inferiority tests used a 2.5% one-sided significance level. All secondary efficacy analyses were performed in an exploratory way using the full analysis population. For analgesic rescue medication intake an additional analysis on per protocol (PP) population was also performed. Safety data were evaluated for all patients who received study drug and for whom at least one post-dose safety assessment was recorded. A subgroup analysis of all cancer patients (with or without cancer-related pain) included in the study was conducted in an exploratory manner providing descriptive statistics and exploratory p-values to compare the treatment groups. All statistical analyses were performed using SAS® version 9.3 or later for Windows software package (SAS Institute, Cary, N.C. 27513).

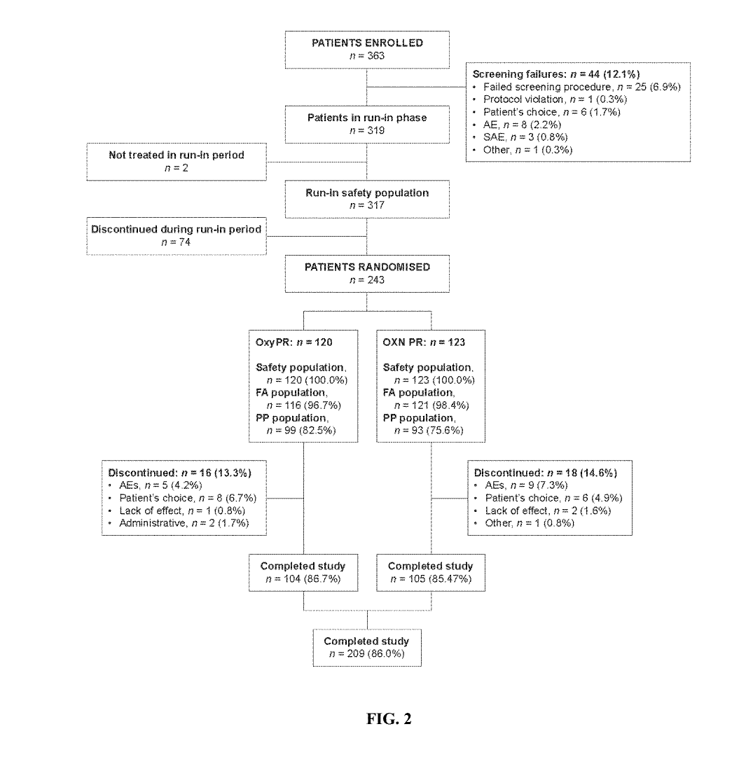

A total of 363 patients were enrolled and screened at 66 clinical centers in 11 countries. Of these, 243 patients were randomly assigned to treatment with OXN PR (n=123) or OxyPR (n=120). In total, 209 patients completed the study: 105 patients in the OXN PR group and 104 patients in the OxyPR group (FIG. 2). After blinded subject evaluability review the following patient populations were available for analysis in the two treatment groups: full analysis, n=121 OXN PR and n=116 OxyPR; per protocol, n=93 OXN PR and n=99 OxyPR; and safety, n=123 OXN PR and n=120 OxyPR. Baseline patient characteristics of the randomised population are shown in Table 1:

TABLE 1
Baseline patient characteristics

| | OXN PR | OxyPR |
|---|---|---|
| Characteristic | (n = 123) | (n = 120) |
| Age, mean (SD), | 57.9 (11.03 [33-86]) | 57.5 (12.33 [21-83]) |
| range (years) | | |
| Gender, n (%) | | |
| Male | 53 (43.1) | 47 (39.2) |
| Female | 70 (56.9) | 73 (60.8) |
| Weight, mean (SD), | 84.7 (21.45 [34-153]) | 83.1 (21.09 [34-165]) |
| range (kg) | | |
| BMI, mean (SD), range | 29.2 (6.52 [14-47]) | 28.3 (6.24 [17-50]) |
| (kg/m²) | | |
| Height, mean (SD), | 170.0 (9.97 [150-196]) | 169.6 (9.87 [150-196]) |
| range (cm) | | |

BMI = body mass index.

Nearly all patients were taking at least one concomitant medication. Just over half of patients (51.0%) were taking medications for disorders of the alimentary tract and metabolism. Consistent with the frequency of musculoskeletal and connective tissue disorders, many patients were taking medications for musculoskeletal symptoms. Non-steroidal anti-inflammatory and anti-rheumatic agents were the most frequently used medications in this therapeutic class.

The number of patients at each dosing level in the two treatment groups were similar, with almost equal proportion of patients at dose levels of 100 mg to 160 mg per day as shown in Table 2:

TABLE 2
Number of patients (%) at each dose
level (full analysis population).

| | |
|---|---|
| Total population | Cancer patients |

| Dose level | OXN PR | OxyPR | OXN PR | OxyPR |
|---|---|---|---|---|
| (mg)^{a} | (n = 121) | (n = 116) | (n = 28) | (n = 22) |
| 100 | 40 (33.1) | 42 (36.2) | 6 (21.4) | 5 (22.7) |
| 120 | 26 (21.5) | 30 (25.9) | 7 (25.0) | 5 (22.7) |
| 140 | 15 (12.4) | 13 (11.2) | 3 (10.7) | 3 (13.6) |
| 160 | 31 (25.6) | 28 (24.1) | 9 (32.1) | 4 (18.2) |
| Other | 9 (7.4) | 3 (2.6) | 3 (10.7) | 5 (22.7) |

^{a}Dose level defined as the highest dose taken on more than 7 consecutive days.
^{b}No specific dose set for more than 7 consecutive days.

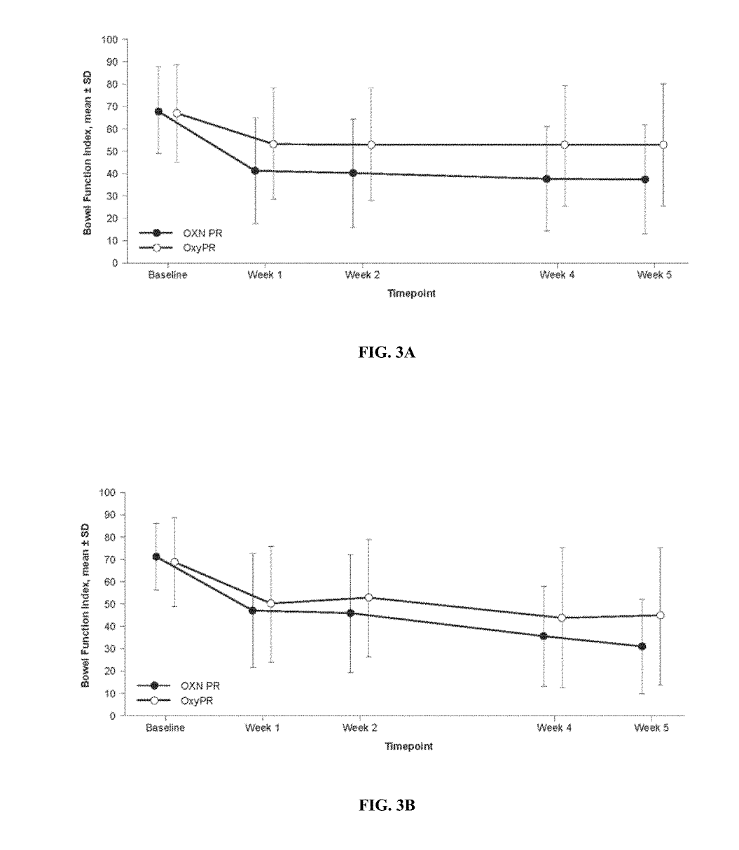

Bowel Function Index—Change in mean BFI scores from baseline values (defined as Visit 3) during the 5 week double-blind phase is shown in FIG. 3A. Reductions in baseline scores were observed beginning at Week 1 and were greater in the OXN PR group compared with the OxyPR group (−28.3 v −13.1). At Week 5 the mean change from baseline continued to be greater in the OXN PR group compared with the OxyPR group (−32.5 v −14.2). Based on the Mixed Model Repeated Measures Analysis (MMRM) the difference between the treatment groups was significant (LS mean difference (SE): −16.05 (3.14); p<0.001, CI: −22.23, −9.86). The difference was also clinically meaningful and relevant being greater than 12 points (Rentz et al., 2009).

The bowel function data were further analyzed based on the different daily amounts of oxycodone administered, namely 100 mg (Table 3), 120 mg (Table 4), 140 mg (Table 5) and 160 mg (Table 6). As for the change in BFI scores from baseline for the above mentioned OXN PR group compared with that of the OxyPR group, the difference in the changes in BFI scores for the two groups were greater than 12 for all tested doses as treatment progressed and particularly by Week 5. In particular, the difference in the change in mean BFI score at Week 5 between the OXN PR group the OxyPR group was −16.1 (−31.7 v. −15.6) for the 100 mg dose, −20.1 (−32.7 v. −12.6) for the 120 mg dose, −16.8 (−37.1 v. −20.3) for the 140 mg dose and −19.9 (−31.2 v. −11.3) for the 160 mg dose. As noted before, a difference of greater than 12 is clinically meaningful and relevant (Rentz et al., 2009). These results include differences significantly greater than 12, e.g., greater than 15, 16, 17, 18, 19 or even 20, and are therefore significantly clinically meaningful.

TABLE 3
Summary of Bowel Function Index (BFI) by Time point-Observed Values: Full Analysis Population/Dosing
Class: 100 mg.

| | | OXN PR (N = 40) | | OxyPR (N = 42) | | Total (N = 82) | |
|---|---|---|---|---|---|---|---|
| | | | Change from | | Change from | | Change from |
| Time point | | Value | Baseline | Value | Baseline | Value | Baseline |
| Screening | n | 40 | | 42 | | 82 | |
| | Mean (SD) | 67.7 (16.40) | | 67.7 (16.80) | | 67.7 (16.50) | |
| | Median | 66.7 | | 70.0 | | 67.5 | |
| | Min, Max | 17, 97 | | 27, 100 | | 17, 100 | |
| Run-in | n | 40 | | 42 | | 82 | |
| | Mean (SD) | 67.4 (17.51) | | 65.8 (17.00) | | 66.6 (17.17) | |
| | Median | 66.7 | | 67.5 | | 66.7 | |
| | Min, Max | 17, 100 | | 30, 100 | | 17, 100 | |
| Baseline | n | 40 | | 42 | | 82 | |
| | Mean (SD) | 64.9 (22.37) | | 61.8 (24.45) | | 63.3 (23.37) | |
| | Median | 70.0 | | 66.7 | | 66.7 | |
| | Min, Max | 0, 100 | | 0, 100 | | 0, 100 | |
| Week 1 | n | 35 | 35 | 41 | 41 | 76 | 76 |
| | Mean (SD) | 40.7 (24.43) | −25.3 (22.43) | 47.4 (27.62) | −13.9 (20.32) | 44.3 (26.24) | −19.1 (21.93) |
| | Median | 43.3 | −20.0 | 53.3 | −3.3 | 49.2 | −10.0 |
| | Min, Max | 0, 100 | −85, 0 | 0, 90 | −77, 7 | 0, 100 | −85, 7 |
| Week 2 | n | 40 | 40 | 41 | 41 | 81 | 81 |
| | Mean (SD) | 38.5 (26.19) | −26.4 (27.10) | 46.0 (28.38) | −15.7 (21.94) | 42.3 (27.41) | −21.0 (25.06) |
| | Median | 38.3 | −21.7 | 50.0 | −6.7 | 46.7 | −13.3 |
| | Min, Max | 0, 97 | −87, 28 | 0, 90 | −83, 15 | 0, 97 | −87, 28 |
| Week 4 | n | 39 | 39 | 40 | 40 | 79 | 79 |
| | Mean (SD) | 34.3 (22.13) | −30.0 (25.01) | 45.3 (28.76) | −16.5 (22.71) | 39.9 (26.12) | −23.2 (24.66) |
| | Median | 30.0 | −25.0 | 50.0 | −10.0 | 43.3 | −16.7 |
| | Min, Max | 0, 90 | −87, 3 | 0, 90 | −77, 12 | 0, 90 | −87, 12 |
| Week 5 | n | 39 | 39 | 38 | 38 | 77 | 77 |
| | Mean (SD) | 34.9 (23.89) | −31.7 (25.67) | 45.4 (28.46) | −15.6 (20.20) | 40.0 (26.60) | −23.8 (24.37) |
| | Median | 33.3 | −30.0 | 50.0 | −10.0 | 43.3 | −16.3 |
| | Min, Max | 0, 90 | −87, 7 | 0, 90 | −77, 12 | 0, 90 | −87, 12 |

TABLE 4
Summary of Bowel Function Index (BFI) by Time point-Observed Values: Full Analysis Population/Dosing
Class: 120 mg.

| | | OXN PR (N = 26) | | OxyPR (N = 30) | | Total (N = 56) | |
|---|---|---|---|---|---|---|---|
| | | | Change from | | Change from | | Change from |
| Time point | | Value | Baseline | Value | Baseline | Value | Baseline |
| Screening | n | 26 | | 30 | | 56 | |
| | Mean (SD) | 70.1 (11.79) | | 72.6 (20.69) | | 71.4 (17.04) | |
| | Median | 68.3 | | 76.7 | | 74.2 | |
| | Min, Max | 43, 97 | | 7, 100 | | 7, 100 | |
| Run-in | n | 26 | | 30 | | 56 | |
| | Mean (SD) | 69.2 (15.70) | | 70.5 (20.05) | | 69.9 (18.02) | |
| | Median | 70.0 | | 71.7 | | 70.0 | |
| | Min, Max | 38, 93 | | 10, 100 | | 10, 100 | |
| Baseline | n | 26 | | 30 | | 56 | |
| | Mean (SD) | 71.4 (15.49) | | 72.9 (16.29) | | 72.2 (15.80) | |
| | Median | 73.3 | | 75.8 | | 73.3 | |
| | Min, Max | 30, 97 | | 40, 100 | | 30, 100 | |
| Week 1 | n | 24 | 24 | 25 | 25 | 49 | 49 |
| | Mean (SD) | 34.6 (21.75) | −36.3 (24.61) | 63.1 (18.22) | −9.3 (21.31) | 49.2 (24.48) | −22.6 (26.53) |
| | Median | 31.7 | −38.3 | 70.0 | −6.7 | 51.7 | −20.0 |
| | Min, Max | 0, 73 | −82, 3 | 20, 93 | −68, 23 | 0, 93 | −82, 23 |
| Week 2 | n | 25 | 25 | 30 | 30 | 55 | 55 |
| | Mean (SD) | 35.4 (26.63) | −36.7 (28.19) | 61.1 (20.36) | −11.8 (17.66) | 49.4 (26.54) | −23.1 (26.04) |
| | Median | 30.0 | −40.0 | 69.2 | −10.8 | 56.7 | −20.0 |
| | Min, Max | 3, 87 | −80, 27 | 20, 92 | −57, 22 | 3, 92 | −80, 27 |
| Week 4 | n | 22 | 22 | 26 | 26 | 48 | 48 |
| | Mean (SD) | 36.9 (24.69) | −36.0 (24.69) | 62.5 (20.78) | −11.5 (18.88) | 50.8 (25.85) | −22.7 (24.79) |
| | Median | 30.8 | −36.7 | 70.0 | −6.7 | 60.0 | −18.3 |
| | Min, Max | 5, 85 | −77, 2 | 18, 93 | −57, 22 | 5, 93 | −77, 22 |
| Week 5 | n | 23 | 23 | 28 | 28 | 51 | 51 |
| | Mean (SD) | 40.2 (28.72) | −32.7 (27.39) | 62.0 (25.69) | −12.6 (26.56) | 52.2 (28.98) | −21.7 (28.51) |
| | Median | 30.0 | −33.3 | 70.0 | −5.8 | 60.0 | −13.3 |
| | Min, Max | 7, 97 | −77, 13 | 5, 100 | −80, 20 | 5, 100 | −80, 20 |

TABLE 5
Summary of Bowel Function Index (BFI) by Time point-Observed Values: Full Analysis Population/Dosing
Class: 140 mg.

| | | OXN PR (N = 15) | | OxyPR (N = 13) | | Total (N = 28) | |
|---|---|---|---|---|---|---|---|
| | | | Change from | | Change from | | Change from |
| Time point | | Value | Baseline | Value | Baseline | Value | Baseline |
| Screening | n | 15 | | 12 | | 27 | |
| | Mean (SD) | 77.8 (19.98) | | 80.7 (14.24) | | 79.1 (17.40) | |
| | Median | 82.3 | | 83.3 | | 83.3 | |
| | Min, Max | 47, 100 | | 53, 100 | | 47, 100 | |
| Run-in | n | 15 | | 13 | | 28 | |
| | Mean (SD) | 78.8 (20.87) | | 72.9 (18.22) | | 76.1 (19.55) | |
| | Median | 85.0 | | 76.7 | | 78.3 | |
| | Min, Max | 37, 100 | | 40, 100 | | 37, 100 | |
| Baseline | n | 15 | | 13 | | 28 | |
| | Mean (SD) | 72.7 (16.54) | | 77.1 (16.86) | | 74.7 (16.53) | |
| | Median | 73.3 | | 83.3 | | 75.8 | |
| | Min, Max | 50, 100 | | 43, 96 | | 43, 100 | |
| Week 1 | n | 13 | 13 | 13 | 13 | 26 | 26 |
| | Mean (SD) | 32.9 (23.39) | −40.9 (33.86) | 55.9 (26.48) | −21.3 (27.08) | 44.4 (27.13) | −31.1 (31.67) |
| | Median | 40.0 | −33.3 | 50.0 | −18.3 | 43.3 | −21.7 |
| | Min, Max | 0, 77 | −100, 3 | 7, 100 | −63, 17 | 0, 100 | −100, 17 |
| Week 2 | n | 15 | 15 | 12 | 12 | 27 | 27 |
| | Mean (SD) | 34.0 (19.83) | −38.7 (31.76) | 58.2 (27.19) | −19.5 (25.46) | 44.8 (25.97) | −30.2 (30.19) |
| | Median | 36.7 | −40.0 | 53.3 | −10.0 | 40.0 | −20.0 |
| | Min, Max | 0, 70 | −100, 10 | 20, 100 | −63, 7 | 0, 100 | −100, 10 |
| Week 4 | n | 14 | 14 | 11 | 11 | 25 | 25 |
| | Mean (SD) | 33.3 (25.00) | −40.7 (33.58) | 50.3 (34.69) | −29.9 (32.40) | 40.8 (30.23) | −36.0 (32.83) |
| | Median | 30.8 | −37.5 | 60.0 | −10.0 | 38.3 | −20.0 |
| | Min, Max | 0, 90 | −100, 0 | 0, 95 | −77, 0 | 0, 95 | −100, 0 |
| Week 5 | n | 14 | 14 | 10 | 10 | 24 | 24 |
| | Mean (SD) | 36.5 (18.23) | −37.1 (27.90) | 61.6 (32.04) | −20.3 (27.25) | 47.0 (27.39) | −30.1 (28.32) |
| | Median | 36.7 | −39.2 | 69.2 | −6.7 | 45.8 | −30.0 |
| | Min, Max | 0, 70 | −85, 17 | 0, 95 | −70, 12 | 0, 95 | −85, 17 |

TABLE 6
Summary of Bowel Function Index (BFI) by Time point-Observed Values: Full Analysis Population/Dosing
Class: 160 mg.

| | | OXN PR (N = 31) | | OXN PR (N = 28) | | OXN PR (N = 59) | |
|---|---|---|---|---|---|---|---|
| | | | Change from | | Change from | | Change from |
| Time point | | Value | Baseline | Value | Baseline | Value | Baseline |
| Screening | n | 31 | | 28 | | 59 | |
| | Mean (SD) | 75.7 (15.81) | | 65.1 (18.55) | | 70.7 (17.84) | |
| | Median | 76.7 | | 66.7 | | 73.3 | |
| | Min, Max | 20, 100 | | 20, 93 | | 20, 100 | |
| Run-in | n | 31 | | 28 | | 59 | |
| | Mean (SD) | 73.5 (15.02) | | 66.0 (18.33) | | 69.9 (16.96) | |
| | Median | 75.0 | | 66.7 | | 71.7 | |
| | Min, Max | 23, 100 | | 20, 93 | | 20, 100 | |
| Baseline | n | 31 | | 28 | | 59 | |
| | Mean (SD) | 69.9 (18.71) | | 61.8 (23.49) | | 66.0 (21.32) | |
| | Median | 70.0 | | 63.3 | | 68.3 | |
| | Min, Max | 10, 97 | | 10, 100 | | 10, 100 | |
| Week 1 | n | 27 | 27 | 24 | 24 | 51 | 51 |
| | Mean (SD) | 49.9 (23.32) | −20.9 (22.63) | 50.4 (23.07) | −11.4 (15.22) | 50.2 (22.97) | −16.4 (19.90) |
| | Median | 53.3 | −16.7 | 50.0 | −6.7 | 53.3 | −10.0 |
| | Min, Max | 0, 90 | −77, 20 | 0, 90 | −53, 7 | 0, 90 | −77, 20 |
| Week 2 | n | 29 | 29 | 28 | 28 | 57 | 57 |
| | Mean (SD) | 48.4 (19.17) | −20.9 (21.91) | 51.7 (21.74) | −10.1 (16.30) | 50.0 (20.36) | −15.6 (19.94) |
| | Median | 53.3 | −16.7 | 55.0 | −6.7 | 53.3 | −13.3 |
| | Min, Max | 0, 83 | −78, 27 | 0, 90 | −37, 23 | 0, 90 | −78, 27 |
| Week 4 | n | 28 | 28 | 26 | 26 | 54 | 54 |
| | Mean (SD) | 43.8 (22.79) | −25.1 (24.34) | 53.2 (23.80) | −8.6 (18.43) | 48.3 (23.54) | −17.2 (23.06) |
| | Median | 41.7 | −23.3 | 58.3 | −6.7 | 50.0 | −18.3 |
| | Min, Max | 0, 93 | −90, 37 | 0, 100 | −53, 23 | 0, 100 | −90, 37 |
| Week 5 | n | 28 | 28 | 25 | 25 | 53 | 53 |
| | Mean (SD) | 37.7 (24.99) | −31.2 (29.10) | 48.6 (22.64) | −11.3 (20.16) | 42.9 (24.31) | −21.8 (26.98) |
| | Median | 31.7 | −25.0 | 50.0 | −10.0 | 46.7 | −15.0 |
| | Min, Max | 0, 87 | −93, 20 | 2, 100 | −73, 27 | 0, 100 | −93, 27 |

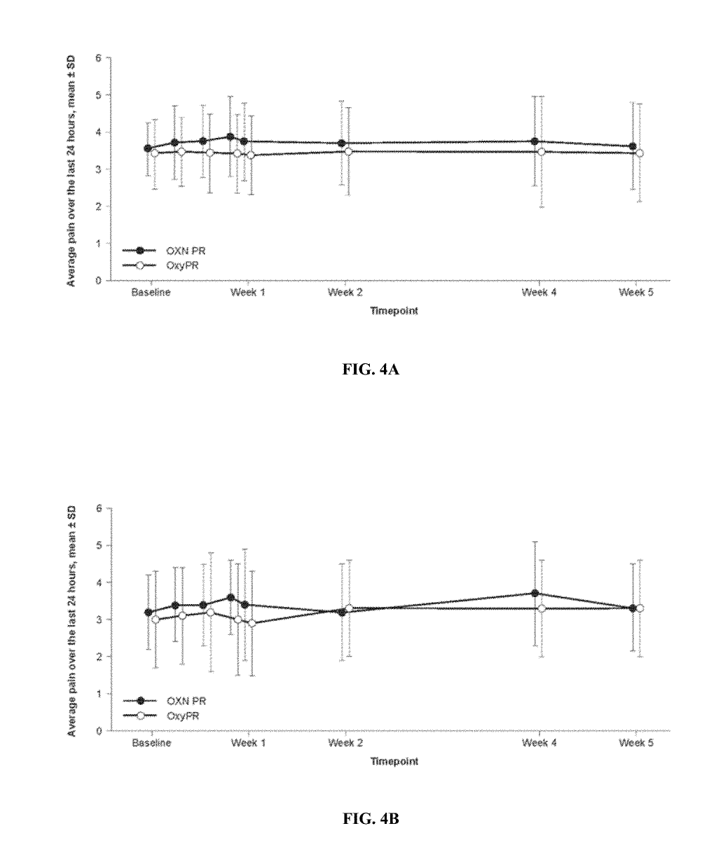

Pain scores—Average 24-hour pain scores were mild and remained stable in the range 3-4 in both treatment groups (FIG. 4A) and non-inferiority of OXN PR to OxyPR was confirmed as statistically significant (p<0.001). Subgroup analyses by dose level showed that subjects receiving 100-120 mg oxycodone per day started with a mean pain score of 4.4 in the OXN PR group and 4.6 in the OxyPR group in the Run-in Phase (Table 7), which was almost 1 score lower than in patients receiving 140-160 mg/d, who had mean pain scores of 5.4 in the OXN PR group and 5.1 in the OxyPR group. However, pain scores at the beginning of the double-blind phase (baseline) and at Week 5 were comparable in the subgroups, which points to a greater level of pain relief in higher dose subgroup.

TABLE 7
Sub-analysis of pain score according to dose of OXN PR or OxyPR received.

| | OXN PR | | OxyPR | |
|---|---|---|---|---|
| | 100-200 mg/d | 140-160 mg/d | 100-200 mg/d | 140-160 mg/d |
| Time point | (n = 60) | (n = 33) | (n = 64) | (n = 35) |
| Run-in (n) | 59 | 33 | 63 | 35 |
| Mean (SD) | 4.4 (1.8) | 5.4 (1.6) | 4.6 (1.9) | 5.1 (1.8) |
| Median (range) | 4.0 (1-10) | 5.0 (2-10) | 4.0 (0-8) | 5.0 (2-9) |
| Baseline (n) | 60 | 33 | 64 | 35 |
| Mean (SD) | 3.5 (0.79) | 3.7 (0.53) | 3.3 (1.03) | 3.5 (0.78) |
| Median (range) | 4.0 (1-5) | 4.0 (2-4) | 4.0 (1-6) | 4.0 (1-4) |
| Week 5 (n) | 60 | 33 | 62 | 32 |
| Mean (SD) | 3.6 (1.29) | 3.6 (0.94) | 3.4 (1.40) | 3.5 (1.19) |
| Median (range) | 4.0 (0-6) | 4.0 (1-6) | 4.0 (0-7) | 4.0 (1-6) |
| Change from baseline (n) | 60 | 33 | 62 | 32 |
| Mean (SD) | 0.2 (1.32) | −0.1 (0.70) | 0.0 (1.34) | 0.1 (0.84) |
| Median (range) | 0.0 (−4-4) | 0.0 (−1-2) | 0.0 (−4-4) | 0.0 (−3-2) |

Analgesic rescue medication—Analgesic rescue medication usage in the per protocol groups is shown in Table 8. Patients took an average of <1 dose and between 10 and 15 mg Oxy IR as analgesic rescue medication per day. No significant differences between treatment groups were detected for frequency of dosing (p=0.5145) or dose (p=0.4328) at Week 5.

TABLE 8
Analgesic and laxative rescue medication use in the per population study groups
for the overall study populations and the cancer sub-group patients.

| | Overall population | | Cancer patients | |
|---|---|---|---|---|
| Time point | OXN PR | OxyPR | OXN PR | OxyPR |
| Analgesic rescue medication, mg/day* | | | | |
| Baseline (n) | 93 | 99 | 17 | 14 |
| Mean (SD) | 11.8 (14.7) | 11.6 (15.7) | 15.1 (19.7) | 11.9 (15.2) |
| Median (range) | 6.4 (0-59) | 4.6 (0-79) | 7.1 (0-59) | 4.5 (0-46) |
| Week 5 (n) | 93 | 99 | 17 | 14 |
| Mean (SD) | 13.8 (16.4) | 14.5 (18.8) | 16.5 (18.3) | 15.2 (17.4) |
| Median (range) | 6.7 (0-68) | 3.0 (0-74) | 14.3 (0-58) | 11.1 (0-50) |
| Laxative rescue medication, mg/day⁺ | | | | |
| Baseline (n) | 121 | 116 | 27 | 19 |
| Mean (SD) | 1.8 (1.5) | 1.5 (1.7) | 1.5 (1.3) | 2.5 (1.7) |
| Median (range) | 1.4 (0-7) | 1.1 (0-9) | 1.4 (0-4) | 2.5 (0-6) |
| Week 5 (n) | 106 | 106 | 21 | 15 |
| Mean (SD) | 0.6 (1.1) | 1.2 (1.7) | 0.6 (1.1) | 1.5 (2.3) |
| Median (range) | 0.0 (0-5) | (0-7) | 0.0 (0-4) | 0.0 (0-6) |

*PP population. Time points are relative to randomisation.
⁺FA population. Time points are relative to first intake of Double-blind IMP.

Laxative rescue medication—Laxative rescue medication use in the full analysis population is also shown in Table 8. Patients receiving OXN PR used significantly lower mean daily doses of laxative rescue medication at Week 5 compared with those receiving OxyPR as the mean (SD) values were 0.6 (1.1) vs 1.2 (1.7) mg/day (p=0.006).

Complete Spontaneous Bowel Movement—The mean number of CSBMs in the full analysis population increased to almost twice the baseline value in the OXN PR group in Week 1 (1.5 to 2.8) while in the OxyPR group a decrease was observed (2.1 to 1.5). An increase in CSBM of 1 is considered clinically relevant. The mean number of CSBMs remained stable through to Week 5 in the OXN PR group at 2.4 compared with 1.4 in the OxyPR group.

EuroQol EQ-5D—The overall EuroQol EQ-5D scores were similar between the two treatment groups and showed a slight increase from Run-in to Week 5. The mean (SD) scores in the OXN PR groups were 0.48 (0.28) and 0.60 (0.25) at baseline and Week 5, respectively. These compare with the equivalent data of 0.45 (0.30) and 0.58 (0.27) for the OxyPR group.

Safety outcomes—Approximately 50% of patients experienced at least one AE in either group (Table 9). Treatment-related AEs with an incidence ≥1% are shown in Table 9. The most common AE in either group was nausea. Four cancer patients died during the study from causes unrelated to the study medication. Nine patients in the OXN PR group and five patients in the OxyPR group discontinued due to AEs.

TABLE 9
Summary of treatment-related adverse events (AEs) occurring
with an incidence of ≥1% (safety population).

| | Overall population | | Cancer patients | |
|---|---|---|---|---|
| | OXN PR | OxyPR | OXN PR | OxyPR |
| | (n = 123) | (n = 120) | (n = 28) | (n = 22) |
| No. AEs | 185 | 143 | 46 | 52 |
| Patients with ≥1 AE, n (%) | 67 (54.5) | 57 (47.5) | 18 (64.3) | 15 (68.2) |
| Patients with ≥1 treatment-related^{a} AE, n (%) | 47 (38.2) | 29 (24.2) | 7 (25.0) | 5 (22.7) |
| No. severe AEs | 10 | 14 | 4 | 9 |
| Patients with ≥1 severe AE, n (%) | 10 (8.1) | 9 (7.5) | 4 (14.3) | 5 (22.7) |
| Patients with ≥1 treatment-related^{a} severe AE, n | 8 (6.5) | 5 (4.2) | 2 (7.1) | 2 (9.1) |
| (%) | | | | |
| Number of SAE | 3 | 6 | 3 | 5 |
| Patients with ≥1 SAE, n (%) | 3 (2.4) | 4 (3.3) | 3 (10.7) | 3 (13.6) |
| Patients with ≥1 treatment-related^{a} SAE, n (%) | 0 | 0 | 0 | 0 |
| Patients who died | 1 (0.8) | 3 (2.5) | 1 (3.6) | 3 (13.6) |
| Most frequent treatment-related^{a} AEs, n (%): | | | | |
| nausea | 10 (8.1) | 5 (4.2) | 2 (7.1) | 1 (4.5) |
| hyperhidrosis | 7 (5.7) | 3 (2.5) | 0 | 0 |
| diarrhea | 5 (4.1) | 4 (3.3) | 0 | 0 |
| upper abdominal pain | 4 (3.3) | 4 (3.3) | 0 | 0 |
| drug withdrawal syndrome | 4 (3.3) | 1 (0.8) | 0 | 0 |
| restlessness | 4 (3.3) | 1 (0.8) | 0 | 0 |
| dizziness | 4 (3.3) | 0 | 2 (7.1) | 0 |

^{a}Investigator considered the AE to be “unlikely”, “possibly”, “probably” or “definitely” related to study medication.
SAE = serious adverse event.
Data are n (%) unless stated otherwise.

Cancer patient sub-analysis—A total of 27 and 19 cancer patients were treated in the OXN PR and OxyPR groups with the number of patients at each dosing level (100 mg to 160 mg per day) being comparable for the two groups (Table 2) analogous to the overall population. Primary endpoint data for BFI (FIG. 3B) and pain scores (FIG. 4B) reveal a similar pattern to that shown for the total population (FIG. 3A and FIG. 4A). A MMRM analysis at Week 5 showed a clinically meaningful and statistically significant treatment difference in BFI of −14.0 (8.1), p=0.047 in favour of OXN PR. Pain scores remained at a low level throughout the study and were comparable between groups. Rescue medication use is shown in Table 8. No significant differences between treatment groups were detected for frequency of analgesic rescue medication intake (p=0.858) or dose (p=0.937) throughout the study. Patients receiving OXN PR used slightly lower mean daily doses of laxative rescue medication (0.8 mg) than those receiving OxyPR (p=0.269). AEs including treatment-related AEs are shown in Table 9. Safety profile was as expected in a population with severe illnesses and a requirement for opioid analgesic treatment in the respective dose range.

### Example 2

Subjects who completed the 5-week Double-blind Phase, or subjects who discontinued the Double-blind Phase prematurely due to constipation, were eligible to enter the Extension Phase, which consisted of additional 24 weeks treatment with open-label OXN PR up to a maximum dose of 90/45 mg twice daily (i.e., 180/90 daily). The extension phase was also open for subjects who required continuation of daily opioid analgesic treatment and were likely to benefit from chronic opioid therapy for the duration of the extension phase. The results of this extension phase are described in Example 2, wherein a particular focus was, in line with Example 1, on the assessment of bowel function and pain.

The Extension Phase duration was up to 24 weeks following the Double-blind Phase plus one additional week Follow-up Period (FIG. 1). In the Extension Phase, Subjects' non-malignant or malignant pain that required around-the-clock opioid therapy was treated with open-label study medication (i.e. OXN PR). All subjects started the Extension Phase with the oxycodone PR dose they had received at the end of the Double-blind Phase. The switch from OXN PR or OxyPR at the end of the Double-blind Phase to open-label OXN PR was done in a stepwise, double-blind, double-dummy manner during the first week of the Extension Phase. Medication titration was permitted at the discretion of the Investigator from Visit 12 onwards. The subject's OXN PR dose could be titrated to a maximum of OXN PR 90/45 mg twice daily. Investigators could prescribe concomitant therapy, including analgesic rescue medication and laxatives, as needed. Oxy IR and bisacodyl were provided only for the first seven days of the Extension Phase. Titration up to the maximum daily dose of OXN PR 90/45 mg twice daily was permitted from Visit 12 onwards. The different dose levels were OXN PR 50/25 mg, OXN PR 60/30 mg, OXN PR 70/35 mg, OXN PR 80/40 mg and OXN PR 90/45 mg twice daily. Almost all subjects who had completed the Double-blind Phase (195 of the 209 subjects) continued to the Extension Phase and 167 subjects (85.6%) completed it.

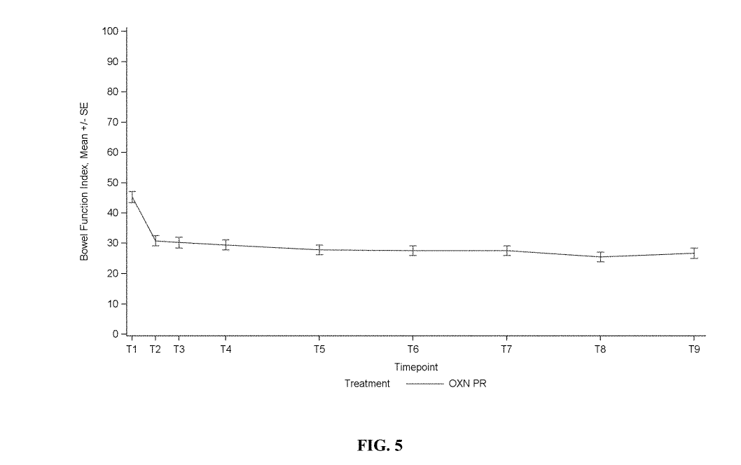

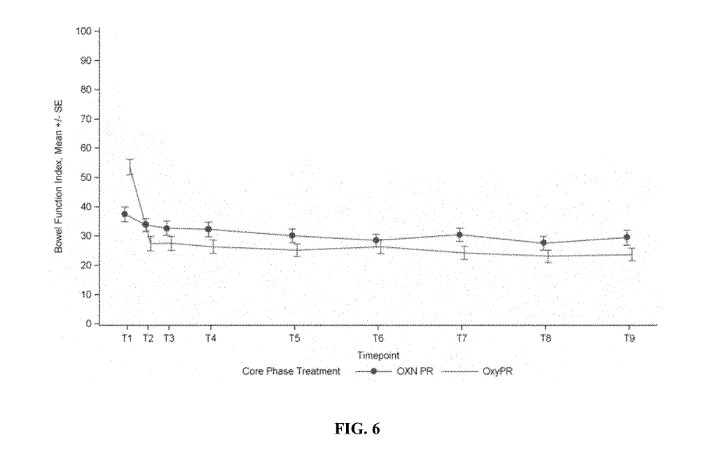

Bowel Function Index—The BFI, which already had improved throughout the Double-blind study, further decreased in the Extension Phase (Table 10, FIG. 5). The greatest decrease was already seen in the first week, when the mean (SD) BFI decreased from 45.3 (26.37) to 30.8 (23.02). The mean (SD) BFI at the end of the study was 26.7 (21.37) and the median was 25.0. Considering that the BFI reference range for non-constipated patients with chronic pain is 0-28.8 (Ueberall et al., 2011), this shows on average a strong tendency for normalization of the bowel function in this study. The subjects, who had received OxyPR in the Double-blind phase started with a higher mean (SD) BFI of 53.6 (25.40) into the Extension Phase (Table 10, FIG. 6). These subjects had a mean (SD) decrease by −25.9 (27.24) in the first week, to a mean (SD) BFI score of 27.5 (23.43). At study end these subjects had a mean (SD) BFI score of 23.7 (19.00), which is a change from baseline of −30.4 (26.15). This was more than twice the clinical relevance limit, which is reached by a decrease of 12 (Rentz et al., 2009). Subjects who had already received OXN PR in the Double-blind Phase, and who already started the Extension Phase with an improved bowel function, showed a further decrease in mean (SD) BFI from 37.5 (24.95) at the start of the Extension Phase to 33.9 (22.32) after the first week and to 29.5 (23.12) at the end of the study. This amounts to an overall decrease of −8.3 (24.77). These results show that the onset of the naloxone effect in patients with opioid-induced bowel dysfunction occurs in the first week of treatment, and an improvement of bowel function continues and is maintained under long-term treatment with OXN PR.

The improvements in bowel function in subjects who initiated OXN PR treatment in the Extension Phase mirrors and confirms the improvement of the bowel function observed in the OXN PR group in the Double-blind Phase, which had decreased in the mean by −28.3 (25.67) in the first week (see Example 1). The improved bowel function—in subjects who were switched to OXN PR at the beginning of the Double-blind Phase as well as subjects who were switched at the beginning of the Extension Phase—improved further and was maintained throughout the duration of the Extension Phase.

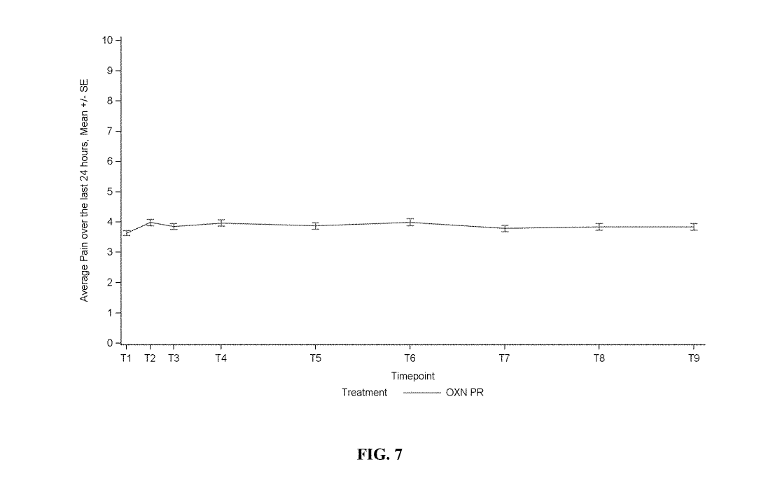

Pain scores—The subjects' average pain over the last 24 hours was measured by the Pain Intensity Scale (NRS 0-10), with 0 meaning no pain and 10 meaning worst imaginable pain. Pain assessments were documented on Visits 11-19. The assessment at Visit 11 served as the baseline for the later pain assessments. The average pain over the last 24 hours remained stable on a low score over the whole period of 24 weeks (FIG. 7). The median pain score was 4.0 throughout the study, in subjects who had received OxyPR in the Double-blind Phase as well as subjects who remained on OXN PR. The overall mean pain score over 24 hours was 3.6 at baseline, and remained between 3.8 and 4.0 throughout the study.

All the subgroups showed the same pattern. No clinically relevant differences were observed between the subgroups, showing comparable analgesic efficacy in younger, older, male and female patients, and in every dose level from 100/50 mg OXN PR to 180/90 mg OXN PR. The pain was stable with minimal changes to baseline. In particular, there was no clinically relevant difference between the dosing subgroups. In the 100-120 mg dose level subgroup the mean (SD) pain score was 3.6 (1.11) at baseline, 3.9 (1.44) after 1 week and 3.7 (1.17) after 24 weeks. In the 140-160 mg dose level subgroup the mean (SD) pain score was 3.7 (1.16) at baseline, 3.9 (1.39) after 1 week and 3.9 (1.45) after 24 weeks. In the >160 mg dose level subgroup the mean (SD) pain score was 3.9 (1.03) at baseline, 4.5 (1.41) after 1 week and 4.4 (1.85) after 24 weeks.

Safety outcomes—A total of 128 subjects (65.6%) experienced 452 AEs, of which 162 in 57 subjects (29.2%) were assessed as causally related to study medication, and 39 in 28 subjects (14.4%) were severe. Twenty-one (21) subjects (10.8%) experienced 36 SAEs, of which 13 SAEs in 6 subjects (3.1%) were assessed as causally related to study medication by the investigator. Four subjects (2.1%) died of unrelated SAEs. More AEs and more related AEs occurred in the week after the switch from OxyPR to OXN PR (70 AEs in 35 subjects, of which 45 AEs in 19 subjects were causally related to study medication) than after the switch from OXN PR in the Double-blind Phase to OXN PR in the Extension Phase (19 AEs in 14 subjects, of which 6 AEs in 5 subjects were causally related to study medication). This was an expected observation, as the onset of the naloxone effect on the intestinal mobility is likely to be associated with gastrointestinal events like diarrhea.

The latter statement is confirmed by the short duration of this effect, as in Weeks 2 to 4 and Weeks 5 to 24, the differences between subjects formerly in the OXN PR and subjects formerly in the OxyPR group had levelled out. In Weeks 2 to 4, 25 subjects (25.0%) previously treated with OXN PR experienced 52 AEs of which 20 were related, and 19 subjects (20.9%) previously treated with OxyPR experienced 36 AEs of which 21 were causally related to study medication. In Weeks 5 to 24, 48 subjects (50.0%) previously treated with OXN PR experienced 142 AEs of which 44 were causally related to study medication, and 42 subjects (47.2%) previously treated with OxyPR experienced 96 AEs of which 20 were causally related to study medication. Despite the greater number of subjects experiencing AEs in Weeks 5 to 24, this points to a decrease in AE rate throughout the study, as the total incidence of AEs declined from 25.1% in the first week to 23.0% over a period of 3 weeks (a rate of 7.7% per week) and to 48.6% over a period of 20 weeks (a rate of 2.4% per week). These results show that long-term use of OXN PR in doses of 100/50 mg to 180/90 mg per day is not associated with an increased safety risk.

The most frequently observed AEs were pain in 16 (8.2%) subjects, diarrhea in 15 (7.7%) subjects, headache in 13 (6.7%) subjects, constipation and nausea in 12 (6.2%) subjects each, and hyperhidrosis in 11 (5.6%) subjects. All these AEs are known adverse reactions of OXN PR and other strong opioids. Pain—though not an AE subsequent to OXN PR administration—is an expected AE in a population with severe pain conditions at any point in time due to fluctuations of their underlying disease. With regards to system organ classes, most AEs were observed in the gastrointestinal disorders SOC, with 50 (25.6%) subjects experiencing 82 gastrointestinal AEs. In the first week 16 (8.2%) subjects experienced gastrointestinal AEs, 13 of whom had been switched from OxyPR. The most frequent AEs in these 13 subjects were diarrhea in 7 subjects, nausea in 4 subjects and upper abdominal pain in 3 subjects. All these AEs point to an onset of the gastrointestinal action of the naloxone within OXN PR in those oxycodone pre-treated subjects. In Weeks 2 to 4, 11 subjects (5.8%) experienced gastrointestinal AEs, of whom 5 were from the former OXN PR and 6 from the former OxyPR group. In Weeks 5 to 24, 26 subjects (14.1%) experienced gastrointestinal AEs, 18 subjects from the former OXN PR group and 8 subjects from the former OxyPR group; this is a mean rate of 0.7% of subjects who experienced gastrointestinal AEs per week. A total of 14 subjects experienced diarrhea (reporting 15 events): 1 subject at 90 mg and at 160 mg, 3 subjects at 100 mg, 3 subjects at 120 mg, 2 subjects at 140 mg, 4 subjects at 160 mg, 1 subject at 180 mg.

TABLE 10
Summary of Bowel Function Index (BFI) by Time point-Observed Values:
Total Exposure Safety Population.

| | | Double-blind Medication | | | | Extension Phase | |
|---|---|---|---|---|---|---|---|
| | | OXN PR | | OxyPR | | Total | |
| | | (N = 100) | | (N = 95) | | (N = 195) | |
| Time | | | Change from | | Change from | | Change from |
| point | | Value | Baseline | Value | Baseline | Value | Baseline |
| Baseline | n | 100 | | 95 | | 195 | |
| (T1) | Mean (SD) | 37.5 (24.95) | | 53.6 (25.40) | | 45.3 (26.37) | |
| | Median | 33.3 | | 58.3 | | 50.0 | |
| | Min, Max | 0, 97 | | 0, 100 | | 0, 100 | |
| Week 1 | n | 100 | 100 | 92 | 92 | 192 | 192 |
| (T2) | Mean (SD) | 33.9 (22.32) | −3.6 (16.09) | 27.5 (23.43) | −25.9 (27.24) | 30.8 (23.02) | −14.3 (24.74) |
| | Median | 30.0 | 0.0 | 26.7 | −20.0 | 26.7 | −5.8 |
| | Min, Max | 0, 95 | −68, 37 | 0, 90 | −90, 23 | 0, 95 | −90, 37 |
| Week 2 | n | 96 | 96 | 89 | 89 | 185 | 185 |
| (T3) | Mean (SD) | 32.7 (24.35) | −3.5 (19.94) | 27.5 (22.95) | −26.6 (28.23) | 30.2 (23.77) | −14.6 (26.84) |
| | Median | 30.0 | 0.0 | 23.3 | −23.3 | 26.7 | −6.7 |
| | Min, Max | 0, 95 | −80, 57 | 0, 94 | −92, 32 | 0, 95 | −92, 57 |
| Week 4 | n | 96 | 96 | 89 | 89 | 185 | 185 |
| (T4) | Mean (SD) | 32.3 (24.24) | −4.1 (21.39) | 26.4 (21.68) | −27.7 (26.54) | 29.5 (23.17) | −15.5 (26.69) |
| | Median | 30.0 | −3.3 | 23.3 | −26.7 | 26.7 | −10.0 |
| | Min, Max | 0, 100 | −77, 52 | 0, 87 | −92, 30 | 0, 100 | −92, 52 |
| Week 8 | n | 96 | 96 | 86 | 86 | 182 | 182 |
| (T5) | Mean (SD) | 30.2 (22.78) | −6.3 (20.52) | 25.2 (19.77) | −28.5 (26.65) | 27.8 (21.50) | −16.8 (26.06) |
| | Median | 26.7 | −3.3 | 23.3 | −29.2 | 25.0 | −11.7 |
| | Min, Max | 0, 90 | −83, 47 | 0, 80 | −92, 30 | 0, 90 | −92, 47 |
| Week 12 | n | 94 | 94 | 84 | 84 | 178 | 178 |
| (T6) | Mean (SD) | 28.5 (20.87) | −7.8 (22.43) | 26.4 (21.95) | −27.4 (30.23) | 27.5 (21.35) | −17.1 (28.09) |
| | Median | 26.7 | −3.3 | 23.3 | −27.5 | 24.2 | −11.7 |
| | Min, Max | 0, 100 | −87, 50 | 0, 90 | −95, 73 | 0, 100 | −95, 73 |
| Week 16 | n | 91 | 91 | 82 | 82 | 173 | 173 |
| (T7) | Mean (SD) | 30.5 (21.74) | −6.6 (23.25) | 24.3 (20.68) | −30.1 (28.98) | 27.6 (21.41) | −17.7 (28.57) |
| | Median | 28.3 | −3.3 | 21.7 | −29.2 | 25.0 | −11.7 |
| | Min, Max | 0, 95 | −83, 57 | 0, 80 | −95, 60 | 0, 95 | −95, 60 |
| Week 20 | n | 89 | 89 | 81 | 81 | 170 | 170 |
| (T8) | Mean (SD) | 27.6 (21.88) | −9.2 (23.83) | 23.2 (19.31) | −31.3 (27.17) | 25.5 (20.75) | −19.7 (27.71) |
| | Median | 23.3 | −5.0 | 21.7 | −30.0 | 23.3 | −14.2 |
| | Min, Max | 0, 97 | −90, 48 | 0, 73 | −95, 27 | 0, 97 | −95, 48 |
| Week 24 | n | 85 | 85 | 79 | 79 | 164 | 164 |
| (T9) | Mean (SD) | 29.5 (23.12) | −8.3 (24.77) | 23.7 (19.00) | −30.4 (26.15) | 26.7 (21.37) | −18.9 (27.68) |
| | Median | 25.0 | −3.3 | 23.3 | −26.7 | 25.0 | −13.3 |
| | Min, Max | 0, 100 | −85, 60 | 0, 80 | −95, 27 | 0, 100 | −95, 60 |

N = Number of subjects in population.
n = Number of subjects with available data.
SD = Standard Deviation.
Time points are relative to first intake of extension phase study medication (IMP).
Baseline defined as Visit 11.

### Example 3

Exemplary suitable tablets are twice-a-day 60/30 and 80/40 OXN tablets. Administration of one of these tablets every 12 hours results in a daily dose of oxycodone of 120 mg (60 mg of naloxone) and of 160 mg (80 mg of naloxone), respectively. The compositions of the tablet cores are given in the following, wherein such cores are usually coated by a cosmetic coating (i.e. the coating will not influence the release of the actives from the tablets).

| | Tablet |
|---|---|
| | 60/30 |
| Ingredient | Amount (mg) |
| Oxycodone HCI, anhydrous | (60.00) |
| corresponding to Oxycodone HCI ¹ | 63.00 |
| Naloxone HCI, anhydrous | (30.00) |
| corresponding to Naloxone | 32.70 |
| hydrochloride dihydrate | |
| Polyvinylpyrrolidone | 14.50 |
| Lactose monohydrate | 77.10 |
| Ethyl cellulose | 24.00 |
| Stearyl alcohol | 59.00 |
| Talc | 5.00 |
| Mg-Stearate | 2.50 |
| Core weight | 277.80 |

¹ calculated based on expected moisture content

| | Tablet |
|---|---|
| | 80/40 |
| Ingredient | Amount (mg) |
| Oxycodone HCI, anhydrous | (80.00) |
| corresponding to Oxycodone HCI ¹ | 84.00 |
| Naloxone HCI, anhydrous | (40.00) |
| corresponding to Naloxone | 43.60 |
| hydrochloride dihydrate | |
| Polyvinylpyrrolidone | 14.50 |
| Lactose monohydrate | 45.20 |
| Ethyl cellulose | 24.00 |
| Stearyl alcohol | 59.00 |
| Talc | 5.00 |
| Mg-Stearate | 2.50 |
| Core weight | 277.80 |

¹ calculated based on expected moisture content

Additional dosage forms suitable for use in the methods described herein are disclosed in WO 2012/020097, the entire contents of which are incorporated by reference.

All patent and non-patent references cited herein are incorporated by reference.

1. A method of improving, maintaining, or normalizing bowel function in a patient on opioid therapy comprising orally administering the patient a daily dose comprising oxycodone or a pharmaceutically acceptable salt thereof and naloxone or a pharmaceutically acceptable salt thereof in a 2:1 weight ratio; wherein
the daily dose comprises at least 90 mg of the oxycodone or pharmaceutically acceptable salt thereof; and
the daily dose is administered as two or more prolonged release oral dosage forms.

2. (canceled)

3. A method of treating pain comprising orally administering to a patient in need thereof a daily dose comprising oxycodone or a pharmaceutically acceptable salt thereof and naloxone or a pharmaceutically acceptable salt thereof in a 2:1 weight ratio; wherein
the daily dose comprises at least 90 mg of the oxycodone or pharmaceutically acceptable salt thereof;
the daily dose is administered as two or more prolonged release oral dosage forms; and
the method improves, maintains, or normalizes the bowel function of the patient.

4. The method of claim 3, wherein the improvement in, maintenance of, or normalization of bowel function is assessed by the difference in bowel function index (BFI) scores following administering the daily dose and a corresponding naloxone-free daily dose, and wherein the difference in BFI scores is at least 12.

5-6. (canceled)

7. The method of claim 4, wherein the difference in BFI scores is at least 18.

8. The method of claim 4, wherein the BFI scores are measured by taking the mean symptom scores of ease of defecation, feeling of incomplete bowel evacuation, and judgment of constipation.

9. The method of claim 3, wherein improving maintaining, or normalizing bowel function comprises reducing or preventing opioid-induced constipation.

10-14. (canceled)

15. A method of treating breakthrough pain in a patient while treating chronic pain and maintaining bowel function in the patient comprising orally administering to the patient immediate release oxycodone without an opioid antagonist for treating breakthrough pain;
wherein the chronic pain is treated by orally administering to the patient a daily dose comprising oxycodone or a pharmaceutically acceptable salt thereof and naloxone or a pharmaceutically acceptable salt thereof in a 2:1 weight ratio;
wherein the daily dose comprises at least 90 mg of the oxycodone or pharmaceutically acceptable salt thereof;
wherein the daily dose is administered as two or more prolonged release oral dosage forms.

16-27. (canceled)

28. The method of claim 3, wherein the patient is suffering from cancer.

29. (canceled)

30. The method of claim 3, wherein subsequent to the method the patient's BFI score is 35 or less.

31-32. (canceled)

33. The method of claim 30, wherein subsequent to the method the patient's BFI score is 29 or less.

34. The method of claim 3, wherein the daily dose comprises 100 mg to 160 mg of the oxycodone or pharmaceutically acceptable salt thereof.

35. The method of claim 3, wherein the daily dose comprises 130 mg to 200 mg of the oxycodone or pharmaceutically acceptable salt thereof.

36. (canceled)

37. The method of claim 3, wherein the daily dose comprises 90 mg, 110 mg, 130, mg, 150 mg, 170 mg, or 190, mg of the oxycodone or pharmaceutically acceptable salt thereof.

38. The method of claim 3, wherein the daily dose comprises 100 mg, 120 mg, 140 mg, 160 mg, 180 mg, or 200 mg of the oxycodone or pharmaceutically acceptable salt thereof.

39. The method of claim 3, wherein the daily dose comprises 120 mg, 160 mg, or 200 mg of the oxycodone or pharmaceutically acceptable salt thereof.

40-49. (canceled)

50. The method of claim 3, wherein the prolonged release oral dosage forms are administered at 12 hour intervals.

51-59. (canceled)

60. The method of claim 3, wherein the daily dose is administered daily for a period of at least 4 weeks.

61. The method of claim 3, wherein the daily dose is administered daily for a period of at least 5 weeks.

62. The method of claim 3, wherein the daily dose is administered daily for a period of at least 8 weeks.

63. The method of claim 3, wherein the daily dose is administered daily for a period of at least 24 weeks.
